(19) United States
(12) Patent Application Publication
(10) Pub. No.: US 2019/0055604 A1
(43) Pub. Date: Feb. 21, 2019

(54) BIOLOGICAL METHODS FOR DIAGNOSING ACTIVE TUBERCULOSIS OR FOR DETEMINING THE RISK OF A LATENT TUBERCULOSIS INFECTION PROGRESSING TO ACTIVE TUBERCULOSIS AND MATERIALS FOR USE THEREIN

(71) Applicant: IMPERIAL INNOVATIONS LIMITED, London (GB)

(72) Inventors: Ajit Lalvani, London (GB); Long Hoang, London (GB); Umar Niazi, London (GB)

(21) Appl. No.: 16/074,540

(22) PCT Filed: Feb. 3, 2017
(86) PCT No.: PCT/GB2017/050275

§ 371 Date: Aug. 1, 2018

(30) Foreign Application Priority Data
Feb. 5, 2016 (GB) 1602142.0

(51) Int. Cl. C12Q 1/6883

(52) U.S. Cl. CPC C12Q 1/6883; C12Q 2600/112; C12Q 2600/106; C12Q 2600/118; C12Q 2600/158

(57) ABSTRACT

There is provided methods for diagnosing active tuberculosis (ATB), or for determining the risk of a latent tuberculosis infection (LTBI) progressing to ATB, in an individual comprising or consisting of the steps of: a) providing a sample to be tested from the individual; b) measuring the expression in the test sample of GBP6 and/or BATF2; wherein the expression in the test sample of GBP6 and/or BATF2 is indicative of the presence of active tuberculosis (ATB) in the individual, and/or wherein the expression in the test sample of GBP6 and/or BATF2 is proportional to the risk of a latent TB infection progressing to an active TB infection in the individual. There are also provided binding agents and kits for use in such methods.

The present invention relates to methods for diagnosing active tuberculosis, or for determining the risk of a latent tuberculosis infection progressing to active tuberculosis, in an individual and binding agents and kits for use in such methods.

Worldwide there are almost 2 million deaths due to active tuberculosis (TB) every year (World Health Organisation (WHO) 2011) and in the developed world, including the UK, rates of TB have risen (Health Protection Agency (HPA) 2011).

A widely used TB diagnostic assay, the mantoux or tuberculin skin test (TST), uses an immune-based approach to demonstrate the presence of infection with Mycobacterium tuberculosis (MTB). The TST has poor specificity especially in those who are BCG-vaccinated (Diel, Goletti et al. 2011) and poor sensitivity especially in those who are immunocompromised e.g. with HIV infection (Cobelens, Egwaga et al. 2006).

The recently introduced interferon-gamma release assays (IGRAs) also use an immune-based approach, which is more quantifiable than the mantoux test and less open to confounding by BCG vaccination or exposure to environmental mycobacteria. Neither the mantoux test nor the IGRAs nor indeed any other commercially available assay can reliably distinguish between latent and active or treated TB, or distinguish ATB from other diseases.

Approximately half of pulmonary TB cases are smear positive for acid fast bacilli (HPA 2011). This method for diagnosing active TB is therefore not very sensitive and is highly disease site specific. The gold standard diagnostic test for active TB is microbiological culture. This was only positive in 58% of cases reported in the UK in 2010 (HPA 2011) and is especially difficult to interpret in paucibacillary disease e.g. lymph node TB, central nervous system TB (CNS TB) and in HIV co-infection. This method of diagnosis is also confined to accessing the site of disease, which can be challenging. The new PCR based approaches e.g. GeneXpert, whilst quicker than microbiological culture cannot distinguish active from latent TB. Both CNS TB and TB with HIV co-infection are associated with high morbidity and mortality. Early and accurate diagnosis is therefore of paramount importance.

Investigation of both active and latent TB infection may involve multiple invasive, time consuming and expensive tests such as CT with contrast, bronchoscopy, biopsy or PET scanning. A blood test that could reduce the need for such intensive investigation would therefore be of great benefit to both patients and healthcare providers.

Treatment of active TB requires a longer, more complex and more toxic treatment regime than the chemoprophylaxis used for LTBI, however both can be associated with potentially life-threatening toxicity. Many therapeutics currently used to treat mycobacterial infection have high associated morbidity e.g. peripheral neuropathy and mortality e.g. liver necrosis. Therefore use of the appropriate shortest and most effective therapeutic regime is vital.

Misdiagnosis of active TB as LTBI can lead to uncontrolled mycobacterial replication and increases the risk of drug resistance, which is more difficult to treat and has a higher associated morbidity and mortality. Misdiagnosis of LTBI and consequent treatment as active TB exposes patients to unnecessary and sometimes toxic therapies for a prolonged period. Furthermore, as most patients with LTBI will never progress to active TB, there is therefore an opportunity to improve care by identifying those most likely to progress and prioritising them to receive treatment. Those unlikely to progress would thus be able to avoid taking chemoprophylaxis altogether.

Currently there is no diagnostic test able to distinguish these two groups. However, given that the likelihood of progression is greatest in the first 2-3 years following MTB infection, correlates of time since exposure may provide a means for risk stratification.

Some data have shown that changes in the antigen-specific cellular immune response mirror pathogen burden and clearance e.g. in cytomegalovirus and HIV infections (Harari, Petitpierre et al. 2004). Recent work on the MTB-specific cellular immune response has suggested that this paradigm holds true in TB (Casey, Blumenkrantz et al.; Millington, Innes et al. 2007). Further studies have focused on using either MTB-specific cell function or cell phenotype to try to distinguish between active and latent infection (Harari, Rozot et al.; Goletti, Butera et al. 2006; Wang, Cao et al. 2010; Mueller, Detjen et al. 2008; Caccamo, Guggino et al. 2009).

Diagnostic tests that are rapid, specific and able to distinguish active Tuberculosis (ATB) from latent TB infection (LTBI) with other concurrent disease (OD) in the clinic are therefore urgently required. Molecular biomarkers that contribute to TB diagnosis in the clinic, particular the challenge of differentiating ATB cases from LTBI with TB like symptoms are still lacking. It has been reported that the combined expression of IL-8, FOXP8, and IL-12b distinguishes ATB from TST+ healthcare workers [1] and expression of FCGR1B, LTF4, CD64 and GBP5 distinguishes ATB from healthy LTBI contacts [2]. Berry et al. [3] identified 86 genes, the majority of which were neutrophil-derived and type-I interferon regulated, distinguishing ATB from other diseases (OD) with 92% sensitivity and 83% specificity, and Kaforou et al. a set of 44 genes differentiating ATB from OD with 100% sensitivity and 96% specificity [4]. However, these case-control studies do not reflect the complexity of TB diagnosis in the clinic, particular the challenge of differentiating ATB cases from LTBI with TB like symptoms. Furthermore complex signatures based on combinations of many genes are difficult to incorporate into routine diagnosis [3, 4].

For example, WO 2011/066008 A2, US 2011/0196614 A1, WO 2014/019977 A1 and WO 2014/093872 A1 each describe methods aimed at diagnosing ATB which rely on complicated gene signatures involving large numbers of genes.

The inventors have now surprisingly found that Guanylate Binding Protein family, member 6 (GBP6) and Basic Leucine zipper Transcription factor, ATF-like 2 (BATF2) are biomarkers of TB which can be used to distinguish ATB from other diseases with or without LTBI and with a high sensitivity and specificity.

Kim et al. (2011) [8] discusses GBP6 (amongst other family members) conferring immunity to bacterial infection such as mycobacterial infection. Roy et al. (2015) [9] describes BATF2 being induced by activated macrophages, such as those infected with M. tuberculosis. However, neither document suggests that such genes may in any way be useful as biomarkers for ATB.

Furthermore, the inventors have identified that each of GBP6 and BATF2 is able to be used alone to distinguish ATB from other diseases with or without LTBI and with a high sensitivity and specificity.

In a first aspect of the invention there is provided a method for diagnosing active tuberculosis (ATB) in an individual comprising or consisting of the steps of:
a) providing a sample to be tested from the individual;
b) measuring the expression in the test sample of GBP6 and/or BATF2;
wherein the expression in the test sample of GBP6 and/or BATF2 is indicative of the presence of active tuberculosis (ATB) in the individual.

GBP6 is an IFNγ-stimulated gene that plays a role in host defence against mycobacteria in mice [7] and in macrophages in vitro, together with GBP1 and GBP10 [7, 8]. BATF2 is strongly induced in IFNγ-activated and Mtb infected macrophages with increased expression of BATF2-dependent genes such as TNF, CCL5, IL12B, and NOS2 [9].

“TB” is used interchangeably herein with “MTB” and “tuberculosis”. It is intended to include TB caused by any MTB specific causing organisms including Mycobacterium Tuberculosis, Mycobacterium Bovis and Mycobacterium africanum. It is particularly preferred that the TB is caused by Mycobacterium Tuberculosis. By “ATB” we mean an active TB infection. By “LTBI” we mean a latent TB infection.

By “sample to be tested”, “test sample” or “control sample” we include a tissue or fluid sample taken or derived from an individual.

By “individual” we refer to any organism capable of being infected with TB. Preferably the individual is a mammal, more preferably selected from cattle, badgers and humans. Most preferably the individual is a human.

Preferably test and control samples are derived from the same species.

The sample may, for example, be a cell or tissue sample (or derivative thereof) comprising or consisting of plasma, plasma cells, serum, tissue cells or equally preferred, protein or nucleic acid derived from a cell or tissue sample.

In preferred embodiments the sample is a blood sample (preferably a PBMC sample).

By “expression” we mean the level or amount (relative and/or absolute) of a gene product such as mRNA or protein.

Methods of detecting and/or measuring the concentration of protein and/or nucleic acids are well known to those skilled in the art, see for example Sambrook and Russell, 2001, Cold Spring Harbor Laboratory Press. In particular, the abundance of nucleic acid such as mRNA can be measured by the polymerase chain reaction (PCR).

The methods described herein may be in vitro methods.

Alternatively or additionally, the method further comprises or consists of the step of diagnosing active ATB in the individual.

In certain embodiments, the method further comprises or consists of the steps of:
c) providing a control sample from an individual not afflicted with active tuberculosis (ATB);
d) measuring the expression in the control sample of GBP6 and/or BATF2;
   wherein the presence of ATB is identified in the event that the expression in the test sample of GBP6 and/or BATF2 measured in step (b) is higher than the expression in the control sample of GBP6 and/or BATF2 measured in step (d).

By “is higher than the expression in the control sample” we mean the expression of GBP6 and/or BATF2 in the test sample is increased over that of the control sample (or increased over predefined reference values representing the same). Preferably, the T-test P value of the difference between the expression in the test sample and control sample is <0.0001 for GBP6 and/or BATF2.

The relative abundance of GBP6 and/or BATF2 can be calculated using the delta-delta Ct method. In brief, for each sample, the amplification value (Ct value) of GBP6 or BATF2 can be measured simultaneously with a house keeping gene (e.g. HPRT1). The expression of HPRT1 was shown to be stable across samples with different clinical condition. Next, the expression of GBP6 or BATF2 in all the samples is normalised to a reference sample (healthy control). Finally, the relative abundance of GBP6 and/or BATF2 in each sample can be used for ROC curve analysis.

For GBP6, a cut-off value of 1.59 can distinguish ATB from OD with AUC value 0.88 (0.84-0.91), and distinguish ATB from LTBI with AUC 0.87 (0.81-0.94).

Similarly, with a cut-off of 3.37, BATF2 can distinguish ATB from OD with AUC 0.86 (0.82-0.89) and distinguish ATB from LTBI with AUC 0.84 (0.77-0.91) (see also Table 1).

In an alternative or additional embodiment the expression in the test sample of GBP6 and/or BATF2 measured in step (b) is significantly higher (i.e., statistically significantly different) from the expression of GBP6 and/or BATF2 measured in step (d) or the predetermined reference values.

For example, significant difference between the presence and/or amount of a particular biomarker in the test and control samples may be classified as those where p<0.0001.

The control sample may comprise or consist of one or more different control samples (e.g. from different individuals). The control sample(s) may be from a healthy individual (i.e., an individual unafflicted by any disease or condition), an individual afflicted with a non-TB disease or condition, or an individual afflicted with a LTBI.

In certain embodiments the method further comprises or consists of the steps of:
e) providing a control sample from an individual afflicted with active tuberculosis (ATB);
f) measuring the expression in the control sample of GBP6 and/or BATF2;
   wherein the presence of ATB is identified in the event that the expression in the test sample of GBP6 and/or BATF2 measured in step (b) corresponds to the expression in the control sample of GBP6 and/or BATF2 measured in step (f).

By “corresponds to the expression in the control sample” we include that the expression of GBP6 and/or BATF2 in the sample to be tested is the same as or similar to the expression of GBP6 and/or BATF2 in the positive control sample. Preferably the expression of GBP6 and/or BATF2 in the sample to be tested is identical to the expression of GBP6 and/or BATF2 in the positive control sample.

Differential expression (up-regulation or down regulation), or lack thereof, can be determined by a well-established methods in the art (e.g. Delta-delta Ct). Differential expression may be determined by a p value of a least less than 0.05 (p=<0.05), for example, at least <0.04, <0.03, <0.02, <0.01, <0.009, <0.005, <0.001, <0.0001, <0.00001 or at least <0.000001.

In one set of embodiments steps (c) and (d) and/or (e) and (f) are performed concurrently with steps (a) and (b).

In other words, steps (c) and (d) may be performed at the same time as (i.e. in parallel with) steps (a) and (b), and/or steps (e) and (f) may be performed at the same time as (i.e. in parallel with) steps (a) and (b), and/or steps (c) and (d) may be performed at the same time as (i.e. in parallel with) steps (e) and (f).

In another set of embodiments steps (c) and (d) and/or (e) and (f) are performed sequentially (i.e. either before or after) with respect to steps (a) and (b).

In other words, steps (c) and (d) may be performed sequentially (i.e. before or after) with respect to steps (a) and (b), and/or steps (e) and (f) may be performed sequentially (i.e. before or after) with respect to steps (a) and (b), and/or steps (c) and (d) may be performed sequentially (i.e. before or after) with respect to steps (e) and (f).

In a further aspect of the invention there is provided a method for determining the risk of a latent tuberculosis infection (LTBI) progressing to active tuberculosis (ATB) comprising or consisting of the steps of:
a) providing a sample to be tested from the individual;
b) measuring the expression in the test sample of GBP6 and/or BATF2;
   wherein the expression in the test sample of GBP6 and/or BATF2 is proportional to the risk of a latent TB infection progressing to an active TB infection in the individual. To confirm this, the expression level of GBP6 and BATF2 is investigated in patients who were the close contacts of ATB cases and were negative with IGRAs test but subsequently progressed to ATB within two years after the samples were collected. The same methodology can be used as for the first aspect.

By proportional we mean positively correlated. For example, a higher expression level of GBP6 and/or BATF2 measured in step (b) correlates with an increased risk of a latent TB infection progressing to an active TB infection.

In another aspect of the invention there is provided a method for distinguishing latent tuberculosis infection (LTBI) from active tuberculosis (ATB) comprising or consisting of the steps of:
a) providing a sample to be tested from the individual;
b) measuring the expression in the test sample of GBP6 and/or BATF2;
   wherein the expression in the test sample of GBP6 and/or BATF2 is higher in patients with ATB relative to those with LTBI.

In certain embodiments of the aspects of the invention the expression of an additional one or more biomarkers is measured alongside the expression of GBP6 and/or BATF2 in step (b), as well as in steps (d) and/or (e) if those steps are being performed.

By “biomarker” we mean a naturally-occurring biological molecule, or component or fragment thereof, the measurement of which can provide information useful in diagnosis of ATB. For example, the biomarker may be a naturally-occurring protein or carbohydrate moiety, or an antigenic component or fragment thereof. By “one or more additional biomarkers” we mean that one or any number of biomarkers may be measured alongside GBP6 and BATF2. In other words the one or more additional biomarker(s) is a different biomarker to GBP6 and BATF2.

The one or more additional biomarkers may advantageously be selected from GBP5, DUSP3, WDFY1, ASGR2, JAK1 and AFF1.

In one set of embodiments the additional one or more biomarkers do not include one or more or all of ANKRD22; FCGR1A; SERPTNG1; FCGR1C; FCGR1B; LOC728744; IFITM3; EPSTI1; GBP5; IF144L; GBP1; LOC400759; IFIT3; AIM2; SEPT4; C1QB; RSAD2; RTP4; IFIT3; CASP5; CEACAM1; CARD 17; ISG15; IFI27; TIMM10; WARS; IFI6; TNFAIP6; PSTPIP2; IFI44; SC02; FBX06; FER1L3; CXCL10; DHRS9; OAS1; STAT1; HP; DHRS9; CEACAM1; SLC26A8; CACNA1E; OLFM4; and APOL6.

In another set of embodiments the additional one or more biomarkers do not include one or more or all of: TMEM144; FBLN5; FBLN5; ERI1; CXCR3; GLUL; LOC728728; KLHDC8B; KCNJ15; RNF125; CCNB1IP1; PSG9; LOC100170939; QPCT; CD177; LOC400499; LOC400499; LOC100134634; TMEM88; LOC729028; EPSTI1; INSC; LOC728484; ERP27; CCDC109A; LOC729580; C2; TTRAP; ALPL; MAEA; COX10; GPR84; TRMT11; ANKRD22; MATK; TBC1D24; LILRA5; TMEM176B; CAMP; PKIA; PFTK1; TPM2; TPM2; PRKCQ; PSTPIP2; LOC129607; APRT; VAMP5; FCGR1C; SHKBP1; CD79B; SIGIRR; FKBP9L; LOC729660; WDR74; LOC646434; LOC647834; RECK; MGST1; PIWIL4; LILRB1; FCGR1B; NOC3L; ZNF83; FCGBP; SNORD13; LOC642267; GBP5; EOMES; BST1; C5; CHMP7; ETV7; ILVBL; LOC728262; GNLY; LOC388572; GATA1; MYBL1; LOC441124; LOC441124; IL12RB1; BRIX1; GAS6; GAS6; LOC100133740; GPSM1; C6orfl29; IER3; MAPK14; PROK1; GPR109B; SASP; LOC728093; PROK2; CTSW; ABHD2; LOC100130775; SLITRK4; FBXW2; RTTN; TAF15; FUT7; DUSP3; LOC399715; LOC642161; LOC100129541; TCTN1; SLAMF8; TGM2; ECE1; CD38; INPP4B; ID3; CR1; CR1; TAPBP; PPAP2C; MBOAT2; MS4A2; FAM176B; LOC390183; SERPING1; LOC441743; H1F0; SOD2; LOC642828; POLB; TSPAN9; ORMDL3; FER1L3; LBH; PNKD; SLPI; SIRPB1; LOC389386; REC8; GNLY; GNLY; FOLR3; LOC730286; SKAP1; SELP; DHX30; KIAA1618; NQ02; ANKRD46; LOC646301; LOC400464; LOC100134703; C20orfl06; SLC25A38; YPEL1; IL1R1; EPHA1; CHD6; LIMK2; LOC643733; LOC441550; MGC3020; ANKRD9; NOD2; MCTP1; BANK1; ZNF30; FBX07; FBX07; ABLIM1; LAMP3; CEBPE; LOC646909; BCL11B; TRIM58; SAMD3; SAMD3; MYOF; TTPAL; LOC642934; FLJ32255; LOC642073; CAMKK2; OAS2; RASGRP1; CAPG; LOC648343; CETP; CETP; CXCR7; UBASH3A; LOC284648; IL1R2; AGK; GTPBP8; LEF1: LEF1; GPR109A; IF135; IRF7; IRF7; SP4; IL2RB; ABLIM1; TAPBP; MAL; TCEA3; KREMEN1; KREMEN1; VNN1; GBP1; GBP1; UBE2C; DET1; ANKRD36; DEFA4; GCH1; IL7R; TMC03; FBX06; LACTB; LOC730953; LOC285296; IL18R1; PRR5; LOC400061; TSEN2; MGC15763: SH3YL1; ZNF337; AFF3; TYMS; ZCCHC14; SLC6A12; LY6E; KLF12; LOC100132317; TYW3: BTLA; SLC24A4; NCALD; ORAI2; ITGB3BP; GYPE; DOCKS; RASGRP4; LOC339290; PRF1; TGFBR3; LGALS9; LGALS9; MGC57346; TXK; DHX58; EPB41L3; LOC100132499: LOC100129674; GDPD5; ACP2; C3AR1; APOB48R; UTRN; SLC2A14; CLEC4D; PKM2; CDCA5; CACNA1E; OSBPL3; SLC22A15; VPREB3; LOC642780; MEGF6; LOC93622; PFAS; LOC729389: CREBZF; IMPDH1; DHRS3; AXIN2; DDX60L; TMTC1; ABCA2; CEACAM1; CEACAM1:FLJ42957; SIAH2; DDAH2; C13orfl 8; TAGLN; LCN2; RELB; NR112; BEND7; PIK3C2B; IF16; DUT; SETD6; LOC100131572; TNRC6A; LOC399744; MAPK13; TAP2; CCDC15; TncRNA; SIPA1L2: HIST1H4E; PTPRE; ELANE; TGM2; ARSD; LOC651451; CYFIP1; CYFIP1; LOC642255; ASCC2; ZNF827; STABI; LMNBI; MAP4K1; PSMB9; ATF3; CPEB4; ATP5S; CD5; SYTL2; H2AFJ; HP: SORT1; KLHL18; HIST1H2BK; KRTAP19-6; RNASE2; LOC100134393; CI Iorf 2; BLK; CD160; LOC100128460; CD19; ZNF438; MBNL3; MBNL3; LOC729010; NAGA; FCER1A; C6orf25: SLC22A4; LOC729686; CTSL1; BCL11A; ACTA2; KIAA1632; UBE2C; CASP4; SLC22A4; SFT2D2: TLR2; C10orfl05; EIF2AK2; TATDN1; RAB24; FAH; DISCI; LOC641848; ARG1; LCK; WDFY3: RNF165; MLKL; LOC100132673; ANKDD1A; MSRB3; LOC100134379; MEFV; C12orf57: CCDC102A; LOC731777; LOC729040; TBC1 D8; KLRF1; KLRF1; ABCA1; LOC650761; LOC653867; LOC648710; SLC2A11; LOC652578; GPR114; MANSCI; MANSCI; DGKA; LIN7A: ITPRIPL2; AN09; KCNJ15; KCNJ15; LOC389386; LOC100132960; LOC643332; SFI1; ABCEI; ABCEI; SERPINA1; OR2W3; ABI3; LOC400759; LOC728519; LOC654053; LOC649553; HSD17B8; C16orB0; GADD45G; TPST1; GNG7; SV2A; LOC649946; LOC100129697; RARRES3; C8orf 3; TNFSF13B; SNRPD3; LOC645232; PI3; WDFY1; LOC100133678; BAMBI; POP5; TARBP1; IRAK3: ZNF7; NLRC4; SKAP1; GAS7; C12orf29; KLRD1; ABHD15; CCDC146; CASP5; AARS2; LOC642103; LOC730385; GAR1; MAF; ARAP2; C16orf7; HLA-C; FLJ22662; DACH1; CRY1; CRY1; LRRC25; KIAA0564; UPF3A; MARCO; SRPRB; MAD1 L1; LOC653610; P4HTM; CCL4L1; LAPTM4B; MAPK14; CD96; TLR7; KCNMB1; P2RX7; LOC650140; LOC791120; LTF; C3orf75; GPX7; SPRYD5; MOV10; EEF1B2; CTDSPL; HIST2H2BE; SLC38A1; AIM2; LOC100130904; LOC650546; P2RY10; IL5RA; MMP8; LOC100128485; RPS23; HDAC7; GUCY1A3; TGFA; NAIP; NAIP; NELL2; SIDTI; SLAMFI; MAPK14; CCR3; MKNKI; D4S234E; NBN; LOC654346; FGFBP2; BTLA; LRRN3; MT2A; LOC728790; LOC646672; NTN3; CD8A; CD8A; ZBP1; LDOC1L; CHM; LOC440731; LOC100131787; TNFRSF10C; LOC651612; STX11; LOC100128060; C1QB; PVRL2; ZMYND15; TRAPPC2P1; SECTM1; TRAT1; CAMKK2; CXCR5; CD163; FAS; RPL12P6; LOC100134734; CD36; FCGR1B; NR3C2; CSGALNACT2; GATA2; EBI2; EBI2; FKBP5; CRISPLD2; LOC152195; LOC100132199; DGAT2; SCMLI; LSS; CIITA; SAP30; TLR5; NAMPT; GZMK; CARD 17; INCA; MSL3L1; CD8A; MIIP; SRPK1; SLC6A6; C10orfl 19; C17orf60; LOC642816; AKR1C3; LHFPL2; CR1; KIAA1026; CCDC91; FAM102A; FAM102A; UPRT; PLEKHA1; CACNA2D3; DDX10; RPL23A; C2orf44; LSP1; C7orf53; DNAJC5; SLAIN1; CDKN1C; HIATL1; CRELD1; ZNHIT6; TIFA; ARL4C; PIGU; MEF2A; PIK3CB; CDK5RAP2; FLNB; GRAP; BATF; CYP4F3; KIR2DL3; C19orf59; NRG1; PPP2R2B; CDK5RAP2; PLSCR1; UBL7; HES4; ZNF256; DKFZp761E198; SAMD14; BAG3; PARP14; MS4A7; ECHDC3; OCIAD2; LOC90925; RGL4; PARP9; PARP9; CD151; SAALI; LOC388076; SIGLEC5; LRIG1; PTGDR; PTGDR; NBPF8; NHS; ACSLI; HK3; SNX20; F2RL1; F2RL1; PARP12; LOC441506; MFGE8; SERPINAIO; FAM69A; IL4R; KIAA1671; OAS3; PRR5; TMEM194; MS4A1; MTHFD2; LOC400793; CEACAM1; APP; RRBP1; SLC04C1; XAFI; XAFI; SLC2A6; ZNF831; ZNF831; POLR1C; GLT1D1; VDR; IFIT5; SNHG8; TOP1MT; UPP1; SYTL2; LOC440359; KLRB1; MTMR3; S1PR1; FYB; CDC20; MEX3C; FAM168B; SLC4A7; CD79B; FAM84B; LOC100134688; LOC651738; PLAGL1; TIMM10; OC641710; TRAF5; TAP1; FCRL2; SRC; RALGAPA1; OCIAD2; PON2; LOC730029; LOC100134768; LOC100134241; LOC26010; PLA2G12A; BACH1; DSC1; NOB1; LOC645693; LOC643313; BTBD11; REPS2; ZNF23: C18orf55; APOL2; APOL2; PASK; FER1L3; U2AF1; LOC285359; SIGLEC14; ARL1; C19orf62; NCR3; HOXB2; RNF135; IFIT1; KLF12; LILRB2; LOC728835; GSN; LOC100008589; LOC100008589; FLJ14213; SH2D3C; LOC100133177; HIST2H2AB; KIAA1618; C21orf2; CREB5; FAS; MTF1; RSAD2; ANPEP; C14orfl79; TXNL4B; MYL9; MYL9; LOC100130828; LOC391019; ITGA2B; KLRC3; RASGRP2; NDST1; LOC388344; IF16; OAS1; OAS1; TRIM10; LIMK2; LIMK2; ATP5S; SMARCD3; PHC2; SOX8; LCK; SAMD9L; EHBP1; E2F2; CEACAM6; LOC100132394; LOC728014; LOC728014; SIRPG; OPLAH; FTHL2; CXorf21; CACNG6; CI lorf75; LY9; LILRB4: STAT2; RAB20; SOCS1; PLOD2; UGDH; MAK16; ITGB3; DHRS9; PLEKHF1; ASAP1IT1; PSME2; LOC100128269; ALX1; BAK1; XP04; CD247; FAM43A; ICOS; ISG15; HIST2H2AA4; CD79A: SLC25A4; TMEM158; GPR18; LAP3; TNFSF13B; TC2N; HSF2; CD7; C20orB; HLA-DRB3; SESN1; LOC347376; P2RY14; P2RY14; P2RY14; CYP1B1; IFIT3; IFIT3; RPL13L; LOC729423; DBN1; TTC27; DPH5; GPR141; RBBP8; LOC654350; SLC30A1; PRSS23; JAM3; GNPDA2; IL7R; ACAD11; LOC642788; ALPK1; LOC439949; BCAT1; ATPGD1; TREML1; PECR; SPATA13; MAN1C1; IDOI; TSEN54; SCRN1; LOC441193; LOC202134; KIAA0319L; MOSC1; PFKFB3; GNB4; ANKRD22: PROS1; CD40LG; RIOK2; AFF1; HIST1H3D; SLC26A8; SLC26A8; RNASE3; UBE2L6; UBE2L6; SSH1; KRBA1; SLC25A23; DTX3L; DOK3; SULT1B1; RASGRP4; ALOX15B; ADM; LOC391825; LOC730234; HIST2H2AA3; HIST2H2AA3; LIMK2; MMRN1; FKBP1A; GYG1; ASF1A; CD248; CD3G; DEFAI; EPHX2; CST7; ABLIM3; ANKRD55; SLC45A3; RAB33B; LILRA6; LILRA6: SPTLC2; CDA; PGD; LOC100130769; ECHDC2; KIF20B; B3GNT8; PYHIN1; LBH; LBH; BPI; GAR1; ST3GAL4; TMEM19; DHRS12; DHRS12; FAM26F; FCRLA; OSBPL7; CTSB; ALDH1A1; SRRD; TOLLIP; ICAM1; LAX1; CASP7; ZDHHC19; LOC732371; DENND1A; EMR2; LOC643308; ADA; LOC646527; LOC643313; GZMB; OLIG2; LA-DPB 1; MX1; THOC3; TRPM6; GK; JAK2; ARHGEF11; ARHGEF11; HOMER2; TACSTD2; CA4; GAA; IFITM3; CLYBL; CLYBL; MME; ZNF408; STAT1; STAT1; PNPLA7; INDO; PDZD8; PDGFD; CTSL1; HOMER3; CEP78; SBK1; ALG9; IL1R2; RAB40B; MMP23B; PGLYRP1; UHRF1; IF144L; PARP10; PARP10; GOLGA8A; CCR7; HEMGN; TCF7; CLUAP1; LOC390735; LOC641849; TYMP; DEFA1B; DEFA1B; DEFA1B; REPS2; REPS2; OSBPLIA; CI lorfl; MCTP2; EMR4; LOC653316; FCRL6; MRPS26; RHOBTB3; DIRC2; CD27; PLEKHG4; CDH6; C4orf23; HIST2H2AC; SLC7A6; SLC7A6; SLAMF6; RETN; FAIM3; TMEM99; LOC728411; TMEM194A; NAPEPLD; ACOX1; CTLA4; SC02; STK3; FLT3LG; VASP; FBX031; TDRD9; TDRD9; LOC646144; NUSAPI; GPR97; GPR97; GPR97; EMRI; SLAMF6; CCDC106; ODF3B; LOC100129904; PADI4; LOC100132858; PIK3AP1; ZNF792; DIP2A; OSCAR; CLIC3; FANCE; TECPR2; P2RY10; ADORA3; IL18RAP; DEFA3; BRSK1; LOC647691; S1PR5CP A3; BMX; DDX58; RHOBTB1; TNFRSF25; LOC730387; OLR1; HERC5; STAT1; NELF; STAP1; ZNF516; ARHGAP26; TIMP2; FCGR1A; RHOH; IF144; MTX3; CD74; LCK; TLR4; DSC2; CXorf45; ENPP4; CD300C; OASL; HPSE; MTHFD2; GSTM2; OLFM4; ABHD12B; LOC728417; LOC728417; FCAR; GTPBP3; KLF4; HOPX; THBD; HIST1H2BG; LOC730995; NOP56; ZBTB9; NLRC3; LOC100134083; COP1; CARD 16; SP140; CD96; POLD2; IL32; LOC728744; FZD2; ZAP70; PYHIN1; SCARF1; IFI27; PFKFB2; PAM; WARS; TCN1; LOC649839; MMP9; TMEM194A; TAP2; C17orf7; LOC728650; PNMA3; CPT1B; LTBP3; CCDC34; PRAGMIN; C9orf1; SMPDL3A; GPR56; C14orfl47; SMARCD3; FAM119A; LOC642334; ENOSF1; FAR2; LOC441763; TESC; CECR6; KIAA1598; GPR109B; LRRN3; RNF213; LRP3; ASGR2; ASGR2; ZSCAN18; MCOLN2; IFIT2; PLCH2; MAP7; GBP4; MGMT; GAL3ST4; C2orf 9; TXNDC3; IFIH1; PRRG4; LOC641693; LOC728093; TNFAIP8L1; AP3M2; BACH2; BACH2; C9orfl23; CACNA1I; LOC100132287; CAMK1D; ANKRD33; CCR6; ALDHIA1; LOC100132797; CD163; ESAM; FCAR; TCN2; CD6; CD3E; CCDC76; MS4A1; IFIT1; MED13L; SLC26A8; NOV; FLJ20035; UGT1A3; LOC653600: LOC642684; KIAA0319L; KLRDI; TRIM22; C4orfl8; TSPAN3; TSPAN3; DNAJC3; AGTRAP; LOC646786; NCALD; TTC25; TSPAN5; ZNF559; NFKB2; LOC652616; HLA-DOA; WARS; GBP2; AUTS2; IGF2BP3; OASL; DYSF; FLJ43093; MS4A14; TGFB111; RAD51C; CALD1; LOC730281; MUC1; C14orfl24; RPL14; APOL6; KCTD12; ITGAX; IFIT3; LPCAT2; ZNF529; AGTRAP; LOC402112; LOC100134822; SH2D1B; MPO; LOC100131967; LOC440459; FAM44B; ACOT9: LOC729915; PDZK1lIP; S100A12; RAB31L1; TMEM204; CXCL10; TSR1; MXD3; LILRA5; CKAP4; C6orfl90; ECGF1; LDLRAP1; GRB10; FCRL3; LOC731275; ZFP91; CTRL; BCL6; SAMD3; LOC647436; CLC; GK; LOC100133565; OAS2; LOC644937; SIRPD; GPBAR1; NL3; CD79B; ELF2; GAA; CD47; NMT2; MATR3; TMEM107; GCM1; RORA; MGAM; LOC100132491; KRT72; SEPT04; ACADVL; ANXA3; MEGF9; MEGF9; PTPRJ; HLA-DRB4; FFAR2; PML; HLA-DQA1; CEACAM8; SH3KBP1; TRPM2; CUXI; LOC648390; SUV39H1; USFI; VAPA; ALOX15; CD79A; DPRXP4; LOC652750; ECM1; ST6GAL1; KLHL3; RTP4; FAM179A; HDC; SACS; C9orf72; C9orf72: LOC652726; PVRIG; PPPIR16B; NSUN7; NSU 7; ZNF783; LOC441013; LOC100129343; OSM; UNC93B1; DNAJC30; FLJ14166; C9orf72; SAMD4A; F5; PARP15; PAFAH2; OL17A1; TYMP; LOC389672; ABCB1; LOC644852; TARP; SLAMF7; FRMD3; LOC648984; PLAUR; LOC100132119; KLRG1; INTS2; MYC; HIST1H4H; C9orf45; KIFAP3; HSPC159; SOCS3; GOLGA8B; LOC100133583; ARL4A; ASNS; ITGAX; LOC153561; GSTMI; OAS2; OAS2; TRIM25; ABHD14A; LOC642342; GPR56; C4orfl 8; AK1; PIK3R6; HSPE1; ASPHD2; DHRS9; GRN; BOAT; LOC100134300; SDSL; TNFAIP6; LOC402176; LOC441019; FAM134B; and ZNF573.

In another set of embodiments, particularly wherein the expression of GBP6 is measured in steps (b), (d) and/or (e), the additional one or more biomarkers do not include one or more or all of: CD79A, CD79B, CXCR5, GNG7, CCR6, ZNF296; C5, FAM20A, DUSP3, GAS6, S100A8, FCGR1B, LHFPL2, FCGR1A, MPO, FCGR1C, GAS6, C1QB, ANKRD22, FCGR1B, C40RF18, C1QC, FLVCR2, VAMP5, SMARCD3, and LOC728744.

In another set of embodiments, particularly wherein the expression of GBP6 is measured in steps (b), (d) and/or (e), the additional one or more biomarkers do not include one or more or all of: C5, FAM20A, DUSP3, GAS6, S100A8, FCGR1B, LHFPL2, FCGR1A, MPO, FCGR1C, GAS6, C1QB, ANKRD22, FCGR1B, C40RF18, C1QC, FLVCR2, VAMP5, SMARCD3, and LOC728744.

In another set of embodiments, particularly wherein the expression of GBP6 is measured in steps (b), (d) and/or (e), the additional one or more biomarkers do not include one or more or all of: HM13BTN3A1, UGP2, CYB561, CYB561, DUSP3, LOC196752, ALDHIAI, PRDMI, CERKL, HM13, RNF19A, MIR1974, PPPDE2, GJA9, CREB5, SERPING1, LOC389386, SEPT_4, RBM12B, CALML4, LHFPL2, CASCI, C190RF12, HLA-DPB1, CD74, ALDHIAI, AAK1, and LOC100133800.

In another set of embodiments, particularly wherein the expression of GBP6 is measured in steps (b), (d) and/or (e), the additional one or more biomarkers do not include one or more or all of: ARG1, IMPA2, RP5-1022P6.2, ORM1, EBF1, PDK4, MAK, VPREB3, HS.131087, MAP7, TMCC1, HS.162734, MAP7, PGA5, HM13BTN3A1, UGP2, CYB561, CYB561, DUSP3, LOC196752, ALDHIAI, PRDMI, CERKL, HM13, RNF19A, MIR1974, PPPDE2, GJA9, CREB5, SERPING1, LOC389386, SEPT_4, RBM12B, CALML4, LHFPL2, CASCI, C190RF12, HLA-DPB1, CD74, ALDHIAI, AAK1, and LOC100133800.

In another set of embodiments, particularly wherein the expression of GBP6 is measured in steps (b), (d) and/or (e), the additional one or more biomarkers do not include one or more or all of: UGP2, BTN3A1, DUSP3, CALML4, FZD2, CYB561, LHFPL2, CYB561, CASCI, RNU4ATAC, VPS13B, PPPDE2, ALDHIAI, GBP5, GAS6, SEP_4, FCGR1B, POLB, CREB5, SIGLECII, LOC389386, DEFA1B, LOC650546, FAM26F, FCGRIA, DEFAIB, ALDHIAI, ANKRD22, IFI27L2, DEFAI, MIR21, DEFA3, FCGRIC, UHMKI, CD74, IL15, and CREG1.

In another set of embodiments, particularly wherein the expression of GBP6 is measured in steps (b), (d) and/or (e), the additional one or more biomarkers do not include one or more or all of: GNG7, BLK, OSBPL10, CXCR5, HEY1, COL9A2, SPIB, LOC90925, ILMN_1916292, EBF1, VPREB3, TMCC1, MAP7, PGA5, ILMN_1893697, UGP2, BTN3A1, DUSP3, CALML4, FZD2, CYB561, LHFPL2, CYB561, CASC1, RNU4ATAC, VPS13B, PPPDE2, ALDHIAI, GBP5, GAS6, SEP_4, FCGR1B, POLB, CREB5, SIGLECII, LOC389386, DEFAIB, LOC650546, FAM26F, FCGRIA, DEFAIB, ALDHIAI, ANKRD22, IFI27L2, DEFAI, MIR21, DEFA3, FCGRIC, UHMKI, CD74, IL15, and CREG1.

In another set of embodiments, particularly wherein the expression of GBP6 is measured in steps (b), (d) and/or (e), the additional one or more biomarkers do not include one or more or all of: LILRB3, PGLYRP1, FAS, IFITM3, FCGR2A, FCGR2A, ST6GAL1, ETS1, CYBB, IFNAR1, LY96, TRIM22, GBP2, DDX58, LAX1, IF116, LCK, IL32, CXCL16, CD40LG, TNFSF13B, IRF2, C5, CD46, TNFAIP6, DPP4, EB12, NFX1, MICB, GBP3, SLAMF7, CARD12, IFIT3, TAP2, HLA-DPB1, CD3G, PRKCQ, IL7R, SLAMF1, CD274, GBP1, IFITM2, ITK, APOL2, PSME1, LAT2, IL18RAP, OSM, CD6, WWP1, CD3E, VIPR1, TNFSF10, PRKRA, TNFRSF1A, BCL6, IL8, OAS3, IFIH1, SIGIRR, SIGIRR, SIT1, ITGAM, C1QB, IL27RA, ALOX5AP, SERPING1, IL1RN, IL1RN, CLEC4D, ICOS, OAS1, ZAP70, IL1B, C4BPA, TNFSF13, IFI30, HPSE, CD59, CTLA4, BCL2, TNFRSF7, FPR1, IL2RA, GATA3, S100A9, TLR8, NCF1, BCL6, BST1, G1P2, C1QA, TCF7, IFITM1, TAPBPL, AIM2, CCR7, LTBR, FYB, NFIL3, LAT, CBLB, CD74, TAP2, FLJ14466, PSMB9, PSMB8, FAIM3, LTA4H, IRF1, OAS2, RELB, TRA@, LTB4R, PIK3R1, OASL, OASL, PSME2, CLEC6A, NBN, FCGR1A, SH2D1A, IL15, LY9, LILRB1, APOL3, PSMB8, CCR6, PDCD1LG2, CD96, EPHX2, BST2, RIPK2, SCAP1, GBP5, TRAT1, ALOX5, LY9, TAP1, RHOH, IFI35, CD28, FYB, IFIT2, TLR7, CD2, FCER1G, SMAD3, FCER1A, SERPINA1, SERPINA1, SECTM1, NMI, TLR5, IFIT3, IFIT3 and CD5.

In another set of embodiments the additional one or more biomarkers do not include one or more or all of: NAIP, AGMAT, CD40LG, PRDM1, LOC730092, FAM102A, KRT72, K1AA0748, MORC2, OASL, CD151, CR1, SPOCK2, SOCS3, DHRS9, P2RY14, BCAS4, MGC22014, RHBDF2, SOCS1, ETS1, KIAA1026, Probe No ILMN_1868912 (Homo sapiens T cell receptor beta variable 21-1, mRNA (cDNA clone MGC:46491 IMAGE:5225843), complete cds), TLR2, LBH, TPM2, TPD52, FCRLA, HLA-DPB1, ABCG1, NAT6, CLUAP1, PASK, ATP6V0E2, POLR1E, MGC42367, HNRPA1L-2, NAIP, ALDH1A1, ID3, ZNF429, SNORD13, CD38, C16orf30, CXCL6, HK2, CLEC4D, SLC30A1, TNFRSF25, OAS2, ASGR2, MAGEE1, LOC642606, KIAA1641, MEF2D, LOC650795, BMX, CXCL10, KCNJ15, LBH, PASK, EVI2A, LIN7A, ETV7, CLEC12A, P2RY14, TXNDC3, NDRG2, CECR6, Probe No ILMN_1915188 (Homo sapiens cDNA FLJ41813 fis, clone NT2RI2011450), DDX58, TIMM10, MYC, SOD2, ISG15, TXNDC12, IF144L, BMX, CDK5RAP2, Probe No ILMN_1823172 (EST10086 human nasopharynx Homo sapiens cDNA, mRNA sequence), FER1L3, IFIT5, KCNJ15, ZAK, Probe No ILMN_1844464 (Human mRNA for T-cell specific protein), ATP8B2, XAF1, C5, GAS6, PIK31P1, SIPA1L2, ANXA3, HIST2H2BF, CR1, ABLIM1, IKZF3, FAM26F, CAPN12, CLEC12A, CDK5RAP2, QPCT, Probe No ILMN_1873034 (Homo sapiens T cell receptor alpha locus, mRNA (cDNA clone MGC:88342 IMAGE:30352166), complete cds), SERPINA1, GAS6, GADD45G, TMEM51, CD274, TSHZ2, LILRA5, CD3D, KIAA1026, B3GNT8, NR3C2, HERC5, OAS3, IL18RAP, LOC653610, GPR109A, LOC728519, TRIM5, LOC642161, TNFRSF25, IF16, TCN2, C11orf1, IGF2BP3, LOC728014, LTB4R, LOC648984, DHRS12, Probe No ILMN_1887868 (Homo sapiens cDNA FLJ20012 fis, clone ADKA03438), ADAM7, BIN1, TCF7, SLC22A4, XRN1, DKFZp761E198, C1QB, LIMK2, LOC653867, IRF7, MMP9, SMARCD3, KLF12, DKFZp761P0423, PVRIG, SOX8, CLYBL, ENTPD1, RSAD2, PARP10, CD27, ABHD14A, OAS1, SATB1, PLSCR1, Probe No ILMN_1889841 (BX092531 NCI_CGAP_Kid5 Homo sapiens cDNA clone IMAGp9981114659; IMAGE:1900882, mRNA sequence), PGLYRP1, LBH, CLEC12A, DHRS12, LIMK2, KREMEN1, FCGBP, PARP9, C9orf66, CD59, EPB41L3, CMPK2, BCL6, LOC648099, C11orf82, CASP5, CCR6, CACNA1E, DHRS9, TNFSF13B, FCAR, C19orf59, GPR109B, FAIM3, Probe No ILMN_1886655 (full-length cDNA clone CS0DI056YK21 of Placenta Cot 25-normalized of Homo sapiens (human), CD5, SRPK1, LOC552891, IL15, IFITM1, ASGR2, Probe No ILMN_1835092 (AGENCOURT_7914287 NIH_MGC_71 Homo sapiens cDNA clone IMAGE:6156595 5, mRNA sequence), GPR141, NOV, PML, CREB5, Probe No ILMN_1860051 (HUMGS0004661 Human adult (K.Okubo) Homo sapiens cDNA 3, mRNA sequence), EPHA4, CDK5R1, LOC652755, ZBP1, LILRB4, URG4, CACNA1I, SELM, OASL, COP1, FRMD3, IL7R, C4orf18, GPR84, ZNF525, EBI2, C12orf57, SLC26A8, C9orf72, GRAP, IFITM3, NELL2, LPCAT2, BLK, IFIT3, AGPAT3, AFF1, PFKFB3, KLF12, IF144, NBN, SLC26A8, OSM, SP140, KIF1B, KLF12, TRIB2, SLC26A8, GNG10, OAS1, Probe No ILMN_1909770 (Homo sapiens cDNA: FLJ21199 fis, clone COL00235), XAF1, LOC650799, IL1RN, DDX60, ECGF1, LIMK2, DOCK9, EBI2, SUCNR1, GZMK, KIAA1618, TNFAIP6, Probe No ILMN_1903064 (BX116726 NCI_CGAP_Pr28 Homo sapiens cDNA clone IMAGp998J065569, mRNA sequence), SERPING1, IFIH1, SIGLECP16, WDFY3, DYSF, CD28, IFIT3, HIST2H2AA3, ADM, ASPHD2, MGC52498, CTSL1, PIK3C2B, SIRPG, ZDHHC19, IFI116, HPSE, EPSTI1, STOM, RAB20, IFI35, SAMD9L, PARP14, LILRA5, IFIT3, GCH1, LMNB1, Probe No ILMN_1819953 (af01b06.sl Human bone marrow stromal cells Homo sapiens cDNA clone IMAGE:1027283 3, mRNA sequence), IFIT2, LAP3, TLR5, TRAFD1, SCO2, TNFSF10, DTX3L, CTSL1, CREB5, HIST2H2AC, SESN1, CEACAM1, ZNF438, C11orf75, HIST2H2AA3, MAPK14, RTP4, LRFN3, PSME1, IL7R, TAP2, FFAR2, KREMEN1, CENTA2, KCNJ15, TRIM5, UBE2L6, FCER1G, PARP9, PRRG4, CASP4, MAFB, APOL1, Probe No ILMN_1845037 (Homo sapiens cDNA clone IMAGE:5277162), GK, CHMP5, ACTA2, TIFA, Probe No ILMN_1859584 (Homo sapiens cDNA: FLJ23098 fis, clone LNG07440), STAT1, SESTD1, STAT2, CEACAM1, SIGLEC5, FCGR1A, LIMK2, ATF3, Probe No ILMN_1851599 (BX110640 Soares_testis_NHT Homo sapiens cDNA clone IMAGp998B094156, mRNA sequence), Sep-04, STAT1, KIAA1618, UBE2L6, HPSE, LACTB, FCGR1B, TRIM22, DRAM, LOC728744, PSTPIP2, AIM2, SLC26A8, FAM102A, FBX06, LOC400759, LHFPL2, GBP1, INCA, GADD45B, DHRS9, LOC440731, SQRDL, ACOT9, TAP1, ANKRD22, C16orf7, PLAUR, MAPK14, GK, GCH1, DYNLT1, FCGR1B, ANKRD22, GBP5, GBP1, PHTF1, WDFY1, GBP2, SRBD1, TAP2, SORT1, PSME2, MAPK14, DHRS9, WARS, WARS, FLVCR2, DUSP3, FER1L3, APOL2, STAT1, BRSK1, JAK2, CEACAM1, GBP4, PSMB9, IL15, MTHFD2, STX11, GYG1, VAMP5, APOL6 and RHBDF2.

Alternatively or additionally, the expression of either GBP6 or BATF2 is not measured as part of the methods of the invention.

In an alternative set of embodiments of the methods of the invention the expression of no more than an additional 1, 2, 3 or 4 biomarkers is measured alongside the expression of GBP6 and/or BATF2. In other words, the expression measured at steps (b), (d) and/or (e) of the methods is that of GBP6 and only 1, 2, 3 or 4 additional biomarkers; or the expression measured at steps (b), (d) and/or (e) of the methods is that of BATF2 and only 1, 2, 3 or 4 additional biomarkers; or the expression measured at steps (b), (d) and/or (e) of the methods is that of GBP6 and BATF2 and only 1, 2 or 3 additional biomarkers.

The additional biomarkers may be selected from GBP5, DUSP3, WDFY1, ASGR2, JAK1 and AFF1.

Alternatively, the additional biomarkers may not include one or more or all of GBP5, DUSP3, WDFY1, ASGR2, JAK1 and AFF1.

In one embodiment of the methods of the invention, the expression of only GBP6 and/or BATF2 is measured in any step. In other words, the expression measured in steps (b), (d) and/or (e) of the methods is of GBP6 and/or BATF2 only and no additional biomarker is measured.

In one embodiment of the methods of the invention, the expression of only GBP6 or BATF2 is measured in any step. In other words, the expression measured in steps (b), (d) and/or (e) of the method is that of GBP6 or BATF2 only and no additional biomarker is measured as part of the method.

In a preferred embodiment of the methods of the invention, the expression of only GBP6 is measured in any step. In other words, the expression measured in steps (b), (d) and/or (e) of the method is of GBP6 only and no additional biomarker is measured as part of the method.

In another preferred embodiment of the methods of the invention, the expression of only BATF2 is measured in any step. In other words, the expression measured in steps (b), (d) and/or (e) of the method is of BATF2 only and no additional biomarker is measured as part of the method.

Measuring the expression of fewer biomarkers as part of the method reduces complexity and thus provides a simpler, easier and more cost-effective method of diagnosis.

In certain embodiments the methods of the invention further comprise or consists of the additional step of performing the Tuberculosis Skin Test (TST) or providing results from a Tuberculosis Skin Test (TST) which has been performed on the individual; optionally wherein the presence of ATB is confirmed in the event that the Tuberculosis Skin Test (TST) or result thereof is positive.

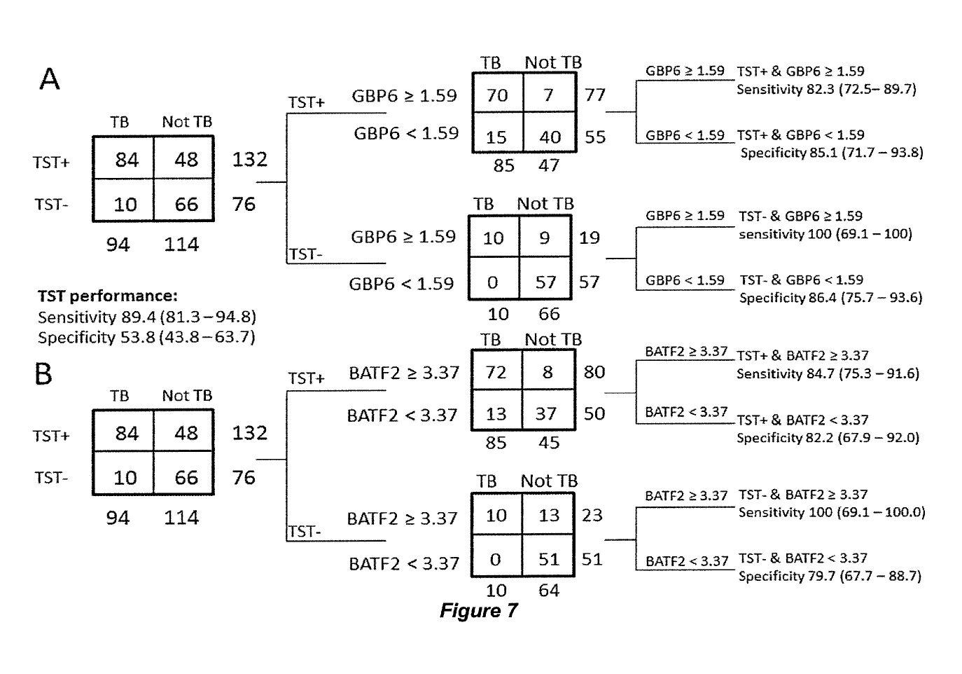

The step of performing the TST may be performed at any time. The TST may be performed concurrently (i.e. in parallel) with the method of the invention, or it may be performed before or after the method of the invention. FIG. 7 details one means of combining the TST with measurement of GBP6 and BATF2.

Alternatively or additionally, IGRA tests such as Quantiferon gold in tube (QTF) and Elispot-based IGRA test (T-SPOT) could be used in combination with the measurement of the expression of GBP6 and/or BATF2 to improve the diagnostic performance of the methods of the invention.

In a preferred embodiment the expression in the test sample of GBP6 is indicative of the presence of active tuberculosis (ATB) in the individual if the relative abundance of expression (e.g. of GBP6 mRNA) is higher than 1.59.

In a preferred embodiment the expression in the test sample of BATF2 is indicative of the presence of active tuberculosis (ATB) in the individual if the relative abundance of expression (e.g. of BATF2 mRNA) is higher than 3.37.

The individual (i.e. the individual from which the sample to be tested derives) may or may not have sarcoidosis. Preferably, the individual does not have sarcoidosis.

Diagnosis of sarcoidosis can be based on exclusion of ATB such as the negative test results for ATB (smear, culture and PCR). Individuals with sarcoidosis often do not respond to TB treatments. In addition, histopathology can be used to investigate the granulomas in sarcoidosis.

The methods of the invention may allow diagnosis of ATB in an individual regardless of whether or not the individual is also infected with HIV. In other words, the methods of the invention may function independently of the HIV status of the individual.

In certain embodiments the individual is infected with HIV. In other embodiments the individual is not infected with HIV, or the HIV infection status of the individual is unknown.

The methods of the invention allow determination of the TB status of an individual regardless of the site of the TB infection. In other words, the methods of the invention function independently of the site of the TB infection. For example, the individual may be or have been infected with TB in the lung (pulmonary) or at any other site (extrapulmonary), or the site of infection may be unknown.

Examples of extrapulmonary infection sites include the pleura (e.g. in tuberculous pleurisy), the central nervous system (e.g. in tuberculous meningitis), the lymphatic system (e.g. in scrofula of the neck), the genitourinary system (e.g. in urogenital tuberculosis), the bones and joints (e.g. in Pott's disease of the spine), the skin, and gastrointestinal manifestations, among others.

In certain embodiments the individual has been infected with TB at any site. In other words, the site of infection may be pulmonary and/or extrapulmonary. In certain embodiments the site of infection is unknown.

In one set of embodiments of the methods of the invention, step (b), (d) and/or step (f) comprises measuring the expression of a nucleic acid molecule encoding GBP6, BATF2 and/or the one or more additional biomarkers.

The nucleic acid molecule may be a cDNA molecule or an mRNA molecule. Preferably, the nucleic acid molecule is an mRNA molecule.

Measuring the expression of the one or more biomarker(s) in step (b), (d) and/or (f) may be performed using, for example, a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably, quantitative real-time PCR (qRT-PCR) is used. Such methods are well known in the art and/or examples are described herein.

Measuring the expression of the GBP6, BATF2 and/or the one or more additional biomarkers in step (b), (d) and/or (f) may be performed using one or more binding moieties, each individually capable of binding selectively to a nucleic acid molecule encoding GBP6, BATF2 or one or more additional biomarkers. The one or more binding moieties may each comprise or consist of a nucleic acid molecule.

The one or more binding moieties each comprise or consist of DNA, RNA, PNA, LNA, GNA, TNA or PMO. Preferably, the one or more binding moieties each comprise or consist of DNA.

The one or more binding moieties may be 5 to 100 nucleotides in length. Preferably, the one or more binding moieties may be 15 to 35 nucleotides in length.

The binding moiety may comprise a detectable moiety. The detectable moiety may be selected from the group consisting of: a fluorescent moiety; a luminescent moiety; a chemiluminescent moiety; a radioactive moiety (for example, a radioactive atom); or an enzymatic moiety.

If the detectable moiety comprises or consists of a radioactive atom, it may be selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Alternatively or additionally, the detectable moiety of the binding moiety may be a fluorescent moiety.

Alternatively or additionally step (b), (d), and/or step (f) of the methods may be performed using a first binding agent capable of binding to the GBP6, BATF2 and/or the one or more additional biomarkers.

Suitable binding agents (also referred to as binding molecules) can be selected from a library, based on their ability to bind a given motif.

At least one type of the binding agents, and more typically all of the types, may comprise or consist of an antibody or antigen-binding fragment of the same, or a variant thereof.

By “antibody” we include substantially intact antibody molecules, as well as chimaeric antibodies, humanised antibodies, human antibodies (wherein at least one amino acid is mutated relative to the naturally occurring human antibodies), single chain antibodies, bispecific antibodies, antibody heavy chains, antibody light chains, homodimers and heterodimers of antibody heavy and/or light chains, and antigen binding fragments and derivatives of the same.

By “antigen-binding fragment” we include a functional fragment of an antibody that is capable of binding to a particular antigen.

The antibody or antigen-binding fragment thereof may be a recombinant antibody or antigen-binding fragment thereof. The antibody or antigen-binding fragment thereof is selected from the group consisting of: scFv; Fab; a binding domain of an immunoglobulin molecule.

The antigen-binding fragment may be selected from the group consisting of Fv fragments (e.g. single chain Fv and disulphide-bonded Fv), Fab-like fragments (e.g.

Fab fragments, Fab′ fragments and F(ab)₂ fragments), single variable domains (e.g. V_{H} and V_{L} domains) and domain antibodies (dAbs, including single and dual formats [i.e. dAb-linker-dAb]).

Also included within the scope of the invention are modified versions of antibodies and an antigen-binding fragments thereof, e.g. modified by the covalent attachment of polyethylene glycol or other suitable polymer.

Methods of generating antibodies and antibody fragments are well known in the art.

For example, antibodies may be generated via any one of several methods which employ induction of in vivo production of antibody molecules, screening of immunoglobulin libraries (Orlandi. et al, 1989. Proc. Natl. Acad. Sci. U.S.A. 86:3833-3837; Winter et al., 1991, Nature 349:293-299) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120).

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

Antibody fragments can be obtained using methods well known in the art (see, for example, Harlow & Lane, 1988, “Antibodies: A Laboratory Manual”, Cold Spring Harbor Laboratory, New York). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

In certain embodiments the antibody or antigen-binding fragment thereof is fixed to a solid support.

In certain embodiments the antibodies or antigen-binding fragments thereof with a particular specificity are separately detectable to those with a different specificity.

In other words an antibody or antigen-binding fragment thereof which is specific for a particular target can be detected as a separate entity to an antibody or antigen-binding fragment thereof which is specific for a different target. In this context ‘target’ refers to any of GBP6, BATF2 or the one or more additional biomarkers.

In a preferred embodiment the antibodies or antigen-binding fragments thereof are visually detectable.

The antibodies or antigen-binding fragments thereof may be labelled in order to allow their detection. For example, they may be labelled with a fluorescent label (e.g. a fluorophore) or with a radio label.

Multiple examples of suitable labels such as fluorophores are well known in the art.

Fluorophores of interest include, but are not limited to fluorescein dyes (e.g. fluorescein dT, 5-carboxyfluorescein (5-FAM), 6-carboxyfluorescein (6-FAM), 2′,4′,1,4,-tetrachlorofluorescein (TET), 2′,4′,5′,7′,1,4-hexachlorofluorescein (HEX), and 2′,7′-dimethoxy-4′,5′-dichloro-6-carboxyfluorescein (JOE)), cyanine dyes such as Cy5, dansyl derivatives, rhodamine dyes (e.g. tetramethyl-6-carboxyrhodamine (TAMRA), ATTO dyes (such as ATTO 647N) and tetrapropano-6-carboxyrhodamine (ROX)), DABSYL, DABCYL, cyanine, such as Cy3, anthraquinone, nitrothiazole, and nitroimidazole compounds, or other non-intercalating dyes.

Preferred possible fluorophores include, amongst others, BD Horizon™ violet laser dyes e.g. BD Horizon™V450, Alexa Fluor® dyes e.g. Alexa Fluor® 488, Fluorescein isothiocyanate (FITC), R-phycoerythrin (PE), PECy™5, PerCP, PerCPCy™5.5, PE-Cy™7, Allophycocyanin (APC), APC-Cy™7, APC-H7, Qdot dyes e.g. Qdot 605.

Particularly preferred fluorophores include, but are not limited to PE-CF594, AF700, QDot605, Qdot655, PE-Cy7, PerCP-Cy5.5, APC-Cy7, BV570, V450, PE, FITC, APC, Biotin, PE-Cy5.

Exemplary fluorophores are used as in the following antibodies used in the methods described herein: LIVE/DEAD® Fixable Dead Cell Stain Kits, aqua, (Invitrogen), CD3-APC-Alexa Fluor®750, CD4-Qdot®605, CD45RA-Qdot®655 (Invitrogen), CD8-APC, CCR7-PE-Cy™7, CD127-FITC (BD Biosciences), PD-1-PerCP/Cy5.5 (Biolegend). IFN-γ-V450, IL-2-PE and TNF-α-AlexaFluor 700 (BD Biosciences)

As used herein, “fluorophore” (also referred to as fluorochrome) refers to a molecule that, when excited with light having a selected wavelength, emits light of a different wavelength.

Alternatively or additionally the first binding agent may be immobilised on a surface (e.g. on a multiwell plate or array).

Antibody fragments may contain one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

A general review of the techniques involved in the synthesis of antibody fragments which retain their specific binding sites is to be found in Winter & Milstein (1991) Nature 349, 293-299.

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic (Clackson et al, 1991, op. cit.; Marks et al, 1991, op. cit.) or eukaryotic cells (Kieke et al, 1999, Proc Natl/Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used often the genes encoding the libraries of potential binding molecules are packaged in viruses and the potential binding molecule is displayed at the surface of the virus (Clackson et al, 1991, op. cit.; Marks et al, 1991, op. cit, Smith, 1985, op. cit.).

The most commonly used such system today is filamentous bacteriophage displaying antibody fragments at their surfaces, the antibody fragments being expressed as a fusion to the minor coat protein of the bacteriophage (Clackson et al, 1991, op. cit.; Marks et al, 1991, op. cit). However, also other systems for display using other viruses (EP 39578), bacteria (Gunneriusson et al, 1999, op. cit.; Daugherty et al, 1998, Protein Eng 11(9):825-32; Daugherty et al, 1999, Protein Eng 12(7):613-21), and yeast (Shusta et al, 1999, J Mol Biol 292(5):949-56) have been used.

In addition, display systems have been developed utilising linkage of the polypeptide product to its encoding mRNA in so called ribosome display systems (Hanes & Pluckthun, 1997, op. cit.; He & Taussig, 1997, op. cit.; Nemoto et al, 1997, op. cit.), or alternatively linkage of the polypeptide product to the encoding DNA (see U.S. Pat. No. 5,856,090 and WO 98/37186).

When potential binding molecules are selected from libraries one or a few selector peptides having defined motifs are usually employed. Amino acid residues that provide structure, decreasing flexibility in the peptide or charged, polar or hydrophobic side chains allowing interaction with the binding molecule may be used in the design of motifs for selector peptides.

For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically, selection of binding agents may involve the use of array technologies and systems to analyse binding to spots corresponding to types of binding molecules.

The variable heavy (V_{H}) and variable light (V_{L}) domains of the antibody are involved in antigen recognition, a fact first recognised by early protease digestion experiments.

Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L}partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

In an alternative or additional embodiment, the first binding agent immobilised on a surface (e.g. on a multiwell plate or array).

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

Hence, the first binding agent may comprise or consist of an antibody or an antigen-binding fragment thereof. Preferably, the antibody or antigen-binding fragment thereof is a recombinant antibody or antigen-binding fragment thereof. The antibody or antigen-binding fragment thereof may be selected from the group consisting of: scFv, Fab, and a binding domain of an immunoglobulin molecule.

Alternatively or additionally the first binding agent comprises or consists of an antibody or an antigen-binding fragment thereof, e.g., a recombinant antibody or antigen-binding fragment thereof. The antibody or antigen-binding fragment thereof may be selected from the group consisting of: scFv; Fab; a binding domain of an immunoglobulin molecule.

Alternatively or additionally the one or more biomarkers in the test sample may be labelled with a detectable moiety.

The detectable moiety may be selected from the group consisting of: a fluorescent moiety; a luminescent moiety; a chemiluminescent moiety; a radioactive moiety; an enzymatic moiety. The detectable moiety may be biotin.

Alternatively or additionally step (b), (d), and/or step (f) may be performed using an assay comprising a second binding agent capable of binding to the one or more biomarkers, the second binding agent comprising a detectable moiety.

The second binding agent may comprise or consist of an antibody or an antigen-binding fragment thereof. The antibody or antigen-binding fragment thereof may be a recombinant antibody or antigen-binding fragment thereof. The antibody or antigen-binding fragment thereof may be selected from the group consisting of: scFv; Fab; a binding domain of an immunoglobulin molecule.

Alternatively or additionally the detectable moiety may be selected from the group consisting of: a fluorescent moiety; a luminescent moiety; a chemiluminescent moiety; a radioactive moiety; an enzymatic moiety. The detectable moiety may be a fluorescent moiety (for example an Alexa Fluor dye, e.g. Alexa647).

Alternatively or additionally the methods of the invention comprise or consist of a qRT-PCR-based assay.

Alternatively or additionally the methods of the invention comprise or consist of an ELISA (Enzyme Linked Immunosorbent Assay).

Alternatively or additionally step (b), (d) and/or step (f) of the method of the invention is performed using an array.

The array may be a bead-based array. The array may be a surface-based array.

Alternatively or additionally the array may be selected from the group consisting of: macroarray; microarray; nanoarray.

In one set of embodiments, the method is 70-100%, 75-100%, 80-100%, 85-100% or 90-100% sensitive. Preferably the method is at least 80% sensitive.

In particular, the sensitivity may be 75.4-86.4% for GBP6 and/or 74.2-85.4% for BATF2. The sensitivity may be improved when TST is used in combination (see FIG. 7).

Alternatively or additionally the method is 70-100%, 75-100%, 80-100%, 85-100% or 90-100% specific. Preferably the method is at least 80% specific.

Sensitivity and specificity are calculated using receiver operator curve analysis.

Alternatively or additionally the predicative accuracy of the method, as determined by an ROC AUC value, is at least 0.80, for example at least 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98 or at least 0.99. Preferably, the predictive accuracy of the method is at least 0.84, 0.85, 0.86, 0.87 or 0.88. The predicative accuracy of the method, as determined by an ROC AUC value, may preferably be at least 0.84 for BATF2 and/or be at least 0.85 for GBP6.

In one set of embodiments, the methods of the invention comprise the additional step of providing the individual with ATB treatment.

The most appropriate ATB treatment may be determined by a Clinician.

For example, the WHO Treatment of Tuberculosis Guidelines (4^{th} Edition, 2010) describes standard treatment regimes for new TB patients. These include treatment with isoniazid, rifampicin, pyrazinamide, ethambutol and/or streptomycin, for example.

In a further aspect there is provided a binding agent specific for GBP6 for determining the presence of ATB (i.e. diagnosing ATB) in an individual, preferably wherein the amount of GBP6 in the individual is indicative of the presence of ATB in the individual.

In a further aspect there is provided a binding agent specific for BATF2 for determining the presence of ATB (i.e. diagnosing ATB) in an individual, preferably wherein the amount of BATF2 in the individual is indicative of the presence of ATB in the individual.

In a further aspect there is provided a binding agent or binding moiety specific for a nucleic acid molecule encoding GBP6 for determining the presence of ATB (i.e. diagnosing ATB) in an individual, preferably wherein the amount of expression of GBP6 in the individual is indicative of the presence of ATB in the individual.

In a further aspect there is provided a binding agent or binding moiety specific for a nucleic acid molecule encoding BATF2 for determining the presence of ATB (i.e. diagnosing ATB) in an individual, preferably wherein the amount of expression of BATF2 in the individual is indicative of the presence of ATB in the individual.

In a further aspect there is provided a use of GBP6 and/or BATF2 for determining the presence of an ATB infection (i.e. diagnosing ATB) in an individual. Optionally, only GBP6 and/or BATF2 alone is used for determining the presence of an ATB infection (i.e. diagnosing ATB) in an individual. Preferably wherein the amount of expression of GBP6 and/or BATF2 in the individual is indicative of the presence of ATB in the individual. The use may be in vitro.

In one embodiment there is provided a use of GBP6 for determining the presence of an ATB infection (i.e. diagnosing ATB) in an individual. Optionally, only GBP6 alone is used for determining the presence of an ATB infection (i.e. diagnosing ATB) in an individual. Preferably wherein the amount of expression of GBP6 in the individual is indicative of the presence of ATB in the individual. The use may be in vitro.

In one embodiment there is provided a use of BATF2 for determining the presence of an ATB infection (i.e. diagnosing ATB) in an individual. Optionally, only BATF2 alone is used for determining the presence of an ATB infection (i.e. diagnosing ATB) in an individual. Preferably wherein the amount of expression BATF2 in the individual is indicative of the presence of ATB in the individual. The use may be in vitro.

In a further aspect there is provided a kit for determining the presence of an ATB infection in an individual comprising or consisting of:
A) one or more first binding agents as defined above, and/or one or more binding moieties as defined above;
B) instructions for performing the method as defined in the first, second or third aspects, or the use as defined in the previous aspect.

Alternatively or additionally, the kit may further comprise a second binding agent as defined above.

In a further aspect of the invention there is provided a method of treating an individual afflicted with ATB comprising performing the method of diagnosis of the first, second or third aspects of the invention and, depending on the diagnosis, administering the most appropriate treatment. The most appropriate treatment may be determined by a Clinician.

Preferences and options for a given aspect, feature or parameter of the invention should, unless the context indicates otherwise, be regarded as having been disclosed in combination with any and all preferences and options for all other aspects, features and parameters of the invention.

Examples embodying an aspect of the invention will now be described with reference to the following figures in which:

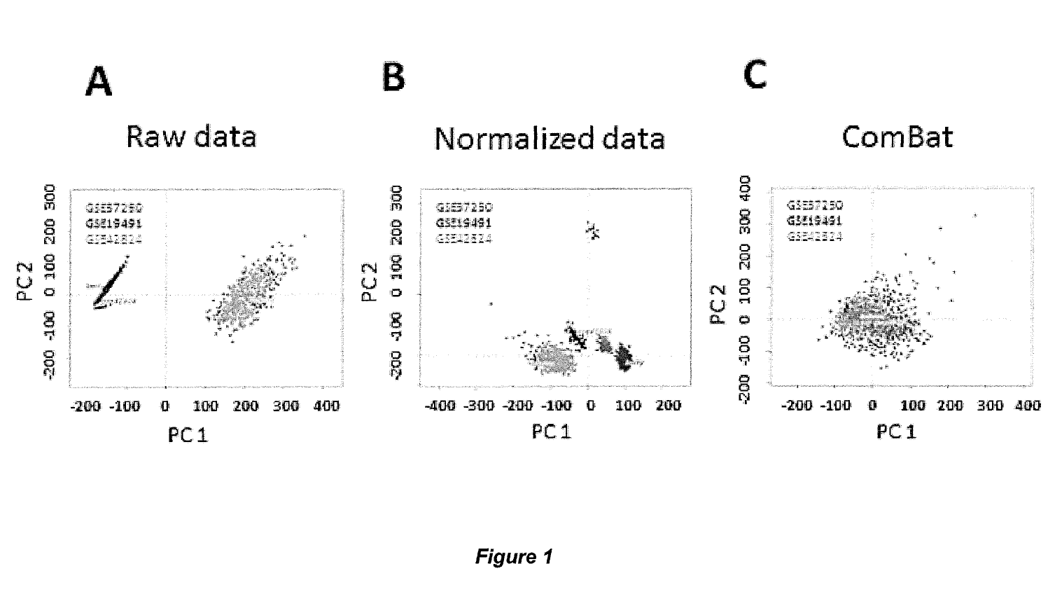

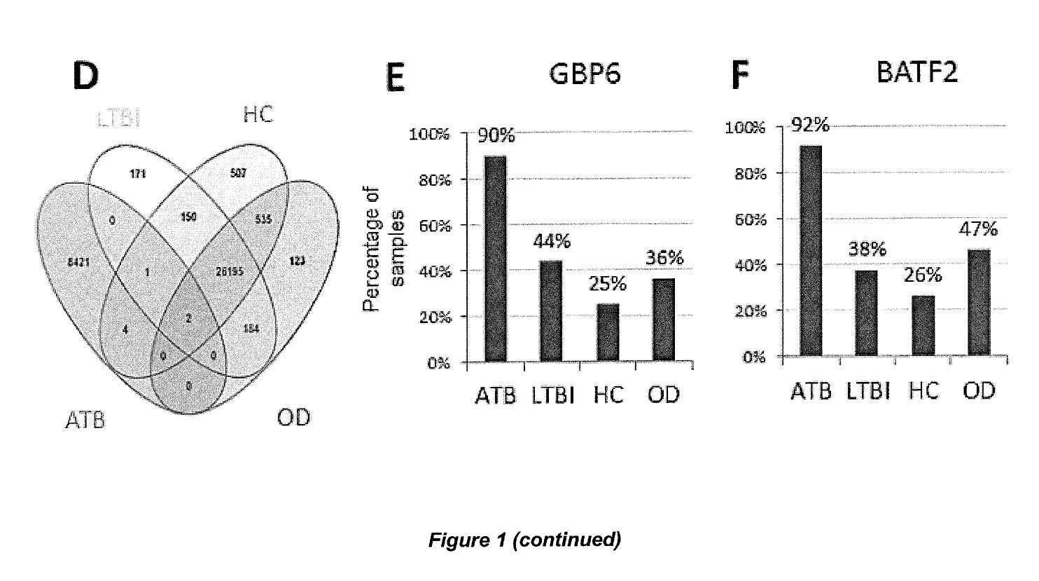

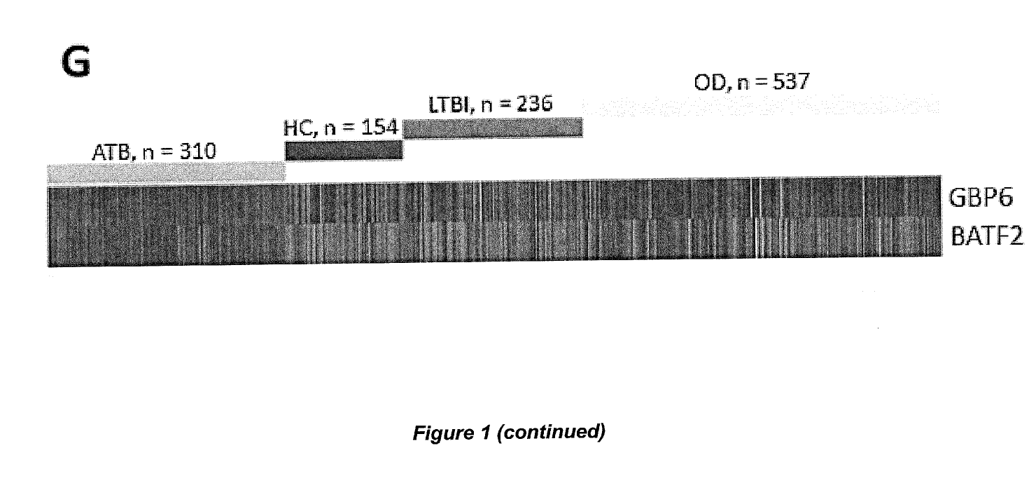

FIG. 1 details data combination and normalisation

(A) Raw expression profiles containing 39,395 transcripts from 1237 subjects were combined. (B) Normalised by Lumi. (C) Batch-effect removed by ComBat. (D) Using detection P values, which are not affected by data combination and normalisation, transcripts that were detected (P value<0.01) in at least 90% of ATB and less than 50% of LTBI, HC (Healthy Control) and OD (Other Diseases) patients were selected. (E) GBP6 was detected in 90% percent of ATB and in only 44% percent, 25% and 36% of LTBI, HC and OD, respectively. (F) Similarly, when HIV+ subjects were removed, BATF2 was detected in 92.9% of ATB patients but in only 37.6%, 26.4% and 46.6% of LTBI, HC and OD respectively. (G) The expression levels of GBP6 and BATF2 were strongly induced in patients with ATB compared with HC, LTBI and OD patients.

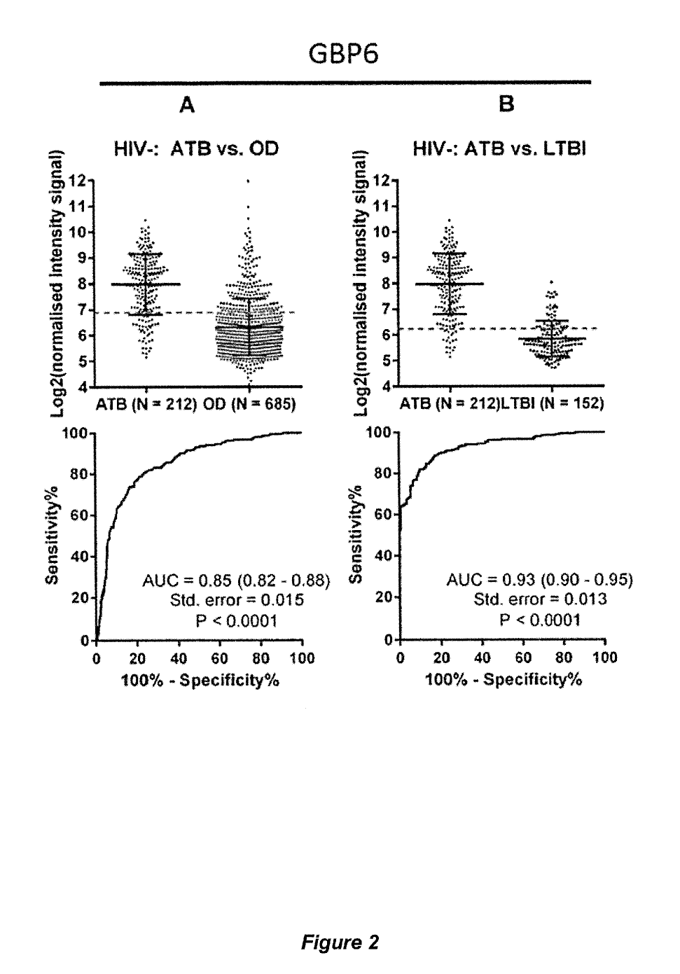

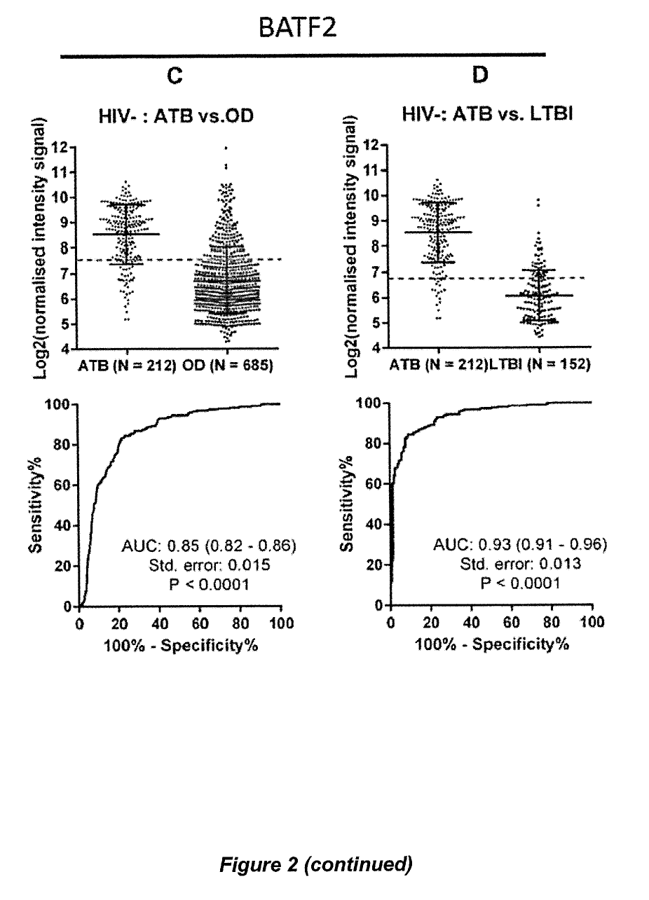

FIG. 2 shows that in HIV− patients, GBP6 and BATF2 were expressed at a significantly higher level in ATB in comparison to OD

(A) ROC curve analysis of GBP6 for distinguishing ATB from OD showing an AUC value of 0.85 (CI 0.82-0.87). (B) GBP6 was expressed at a significantly higher level in ATB in comparison with LTBI and a high AUC value of 0.93 (0.90-0.95) was observed in ROC curve analysis. (C) ROC curve analysis of BATF2 for distinguishing ATB from OD showing a AUC value of 0.85 (CI 0.82-0.86). (D) BATF2 was expressed at a significantly higher level in ATB in comparison with LTBI and a high AUC value of 0.93 (0.91-0.96) was observed in ROC curve analysis. The dashed line indicates the cut-off value. The sensitivity, specificity, positive likelihood ratio and negative likelihood ratio values associated with this cut-off value are summarised in Table 2.

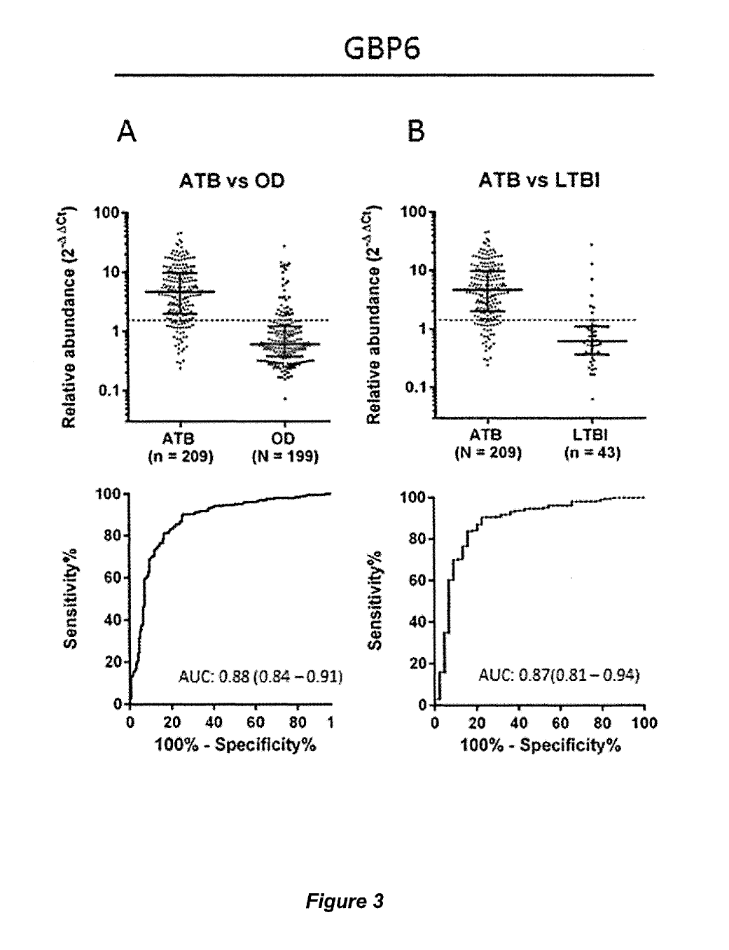

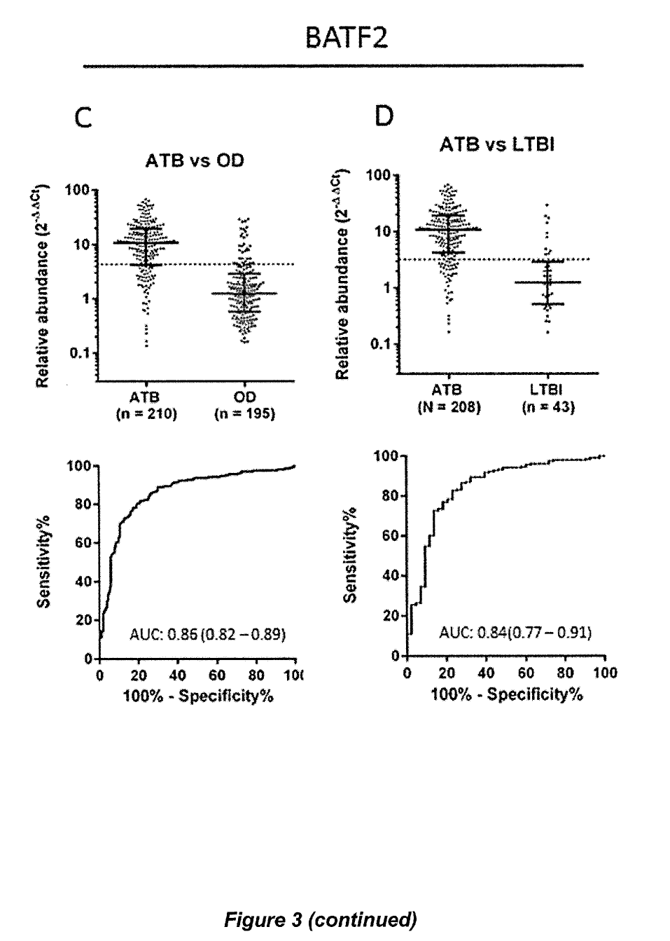

FIG. 3 shows the diagnostic performance of GBP6 and BATF2 in distinguishing ATB

Diagnostic performance of GBP6 (A & B) and BATF2 (C & D) in distinguishing ATB from OD and LTBI was validated by quantitative PCR in an IDEA (IGRA in Diagnostic Evaluation of Active TB) patient cohort. GBP6 was expressed at a significantly higher level in patients with ATB (N=209) relative to those with OD (N=199) (A) or with LTBI (N=43) (B). An AUC value of 0.88 (0.84-0.91) was observed in ROC curve analysis of GBP6 for discriminating ATB from OD (A) and GBP6 could also differentiate ATB from LTBI with an AUC value of 0.87 (0.81-0.94) (B). BATF2 was expressed at a significantly higher level in patients with ATB (N=209) relative to those with OD (N=195) (C) or with LTBI (N=43) (D). An AUC value of 0.86 (0.82-0.89) was observed in ROC curve analysis of BATF2 for discriminating ATB from OD (C) and BATF2 could also differentiate ATB from LTBI with an AUC value of 0.84 (0.77-0.91) (D). The details of ROC curve analysis with sensitivity, specificity and likelihood ratio are summarised in Table 1.

TABLE 1
Validation of GBP6 and BATF2 expression profiles by qPCR

| | | AUC | Sensitivity | Specificity | LR+ | LR− |
|---|---|---|---|---|---|---|
| | | (95CI) | (95CI) | (95CI) | (95CI) | (95CI) |
| GBP6 | ATB vs. | 0.85 | 81.3 | 78.1 | 3.7 | 0.23 |
| | OD* | (0.81-0.88) | (75.4-86.4) | (72.1-83.4) | (2.7-5.2) | (0.16-0.34) |
| | ATB vs. | 0.88 | 81.3 | 83.4 | 4.9 | 0.22 |
| | OD^{‡} | (0.84-0.91) | (75.4-86.4) | (77.5-88.3) | (3.4-7.4) | (0.15-0.31) |
| | ATB vs. | 0.87 | 83.7 | 84.1 | 5.3 | 0.19 |
| | LTBI^{†} | (0.81-0.94) | (78.0-88.5) | (69.9-93.4) | (2.6-13.4) | (0.12-0.31) |
| BATF2 | ATB vs. | 0.84 | 80.3 | 75 | 3.2 | 0.26 |
| | OD* | (0.80-0.88) | (74.2-85.5) | (68.7-80.6) | (2.4-4.4) | (0.18-0.37) |
| | ATB vs. | 0.86 | 80.3 | 80 | 4 | 0.24 |
| | OD^{‡} | (0.82-0.89) | (74.2-85.5) | (73.7-85.4) | (2.8-5.9) | (0.17-0.35) |
| | ATB vs | 0.84 | 82.2 | 76.7 | 3.5 | 0.23 |
| | LTBI^{‡} | (0.77-0.91) | (76.3-85.5) | (61.4-88.2) | (2.0-7.2) | (0.16-0.38) |

*Including patients with sarcoidosis (n = 25);
^{‡}without sarcoidosis (n = 25),
^{†}Double positive (TST and QTF or TST and TSPOT or QTF and TSPOT)

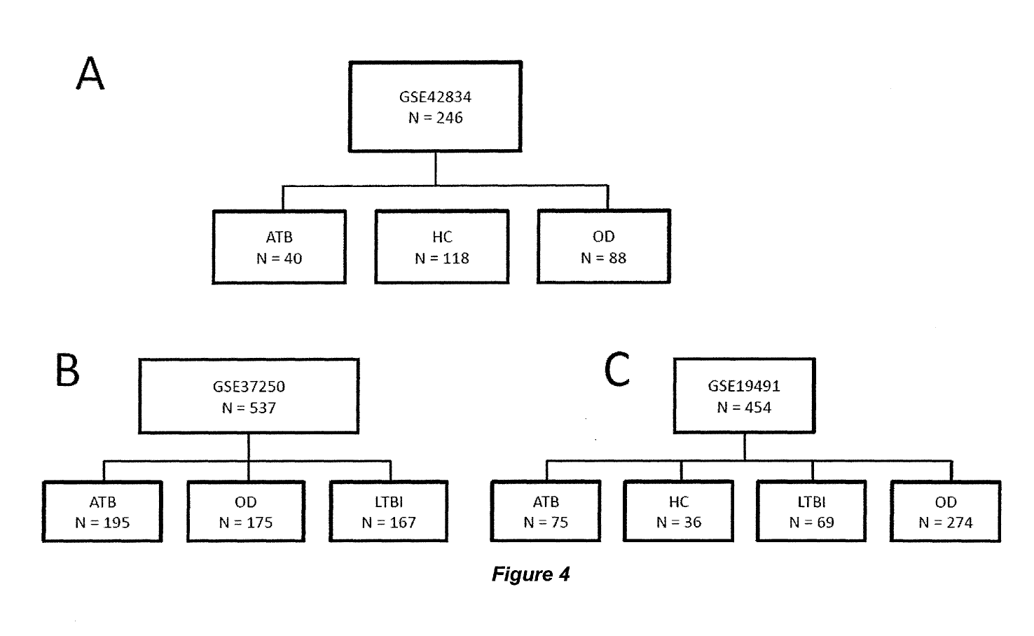

FIG. 4 shows a summary of samples from previous published studies used for analysis

A total of 1237 samples were retrieved. Amongst these, 310 were ATB, 236 were LTBI, 152 were healthy controls and 537 were diagnosed with other diseases. LTBI subjects from these studies were recruited from healthy people who participated in HIV screening programmes in Africa.

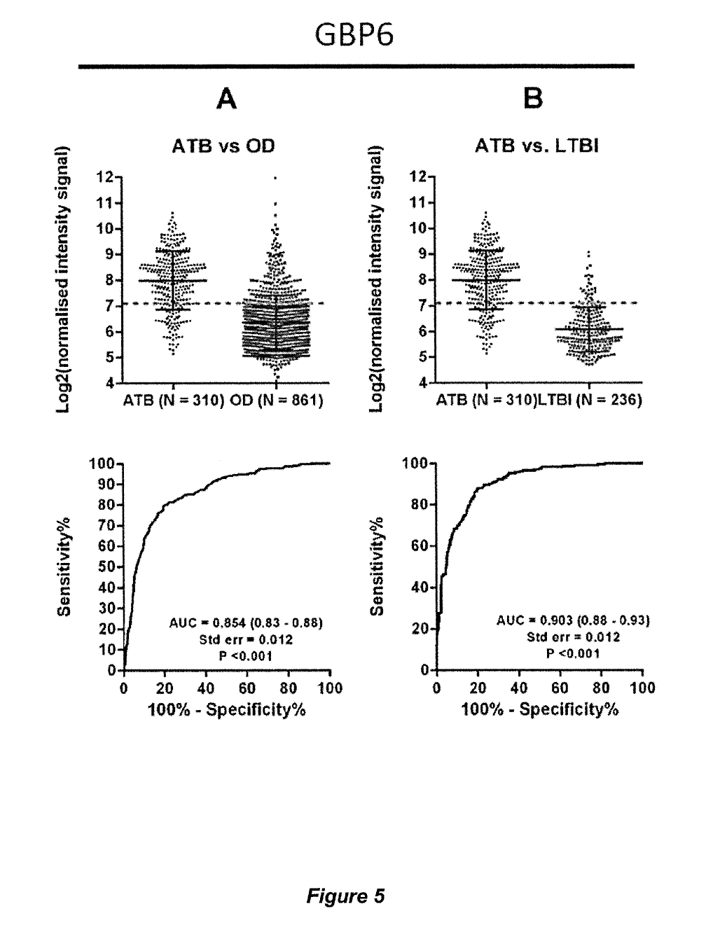

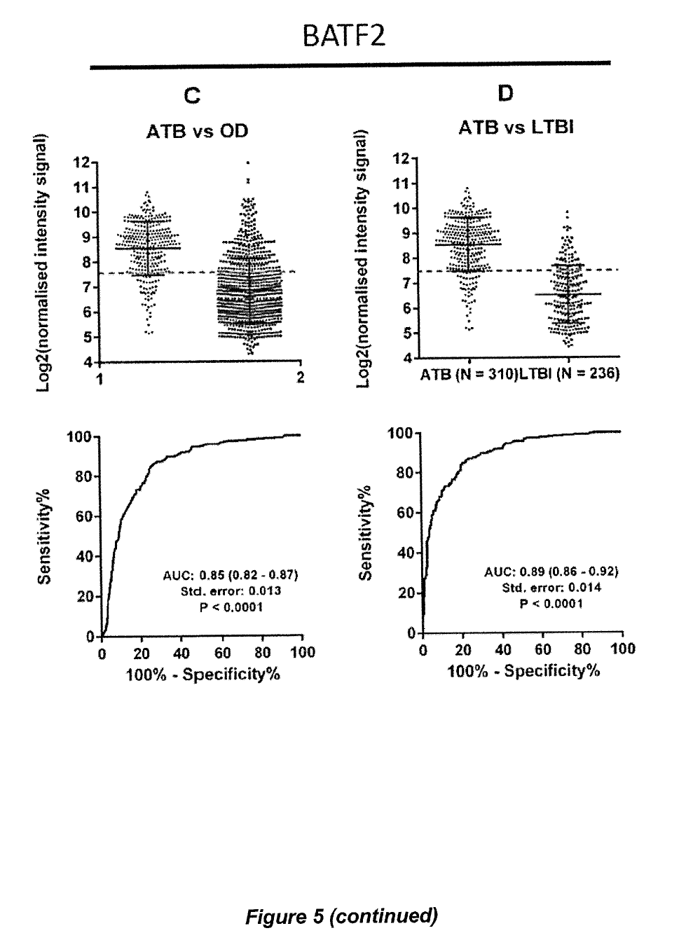

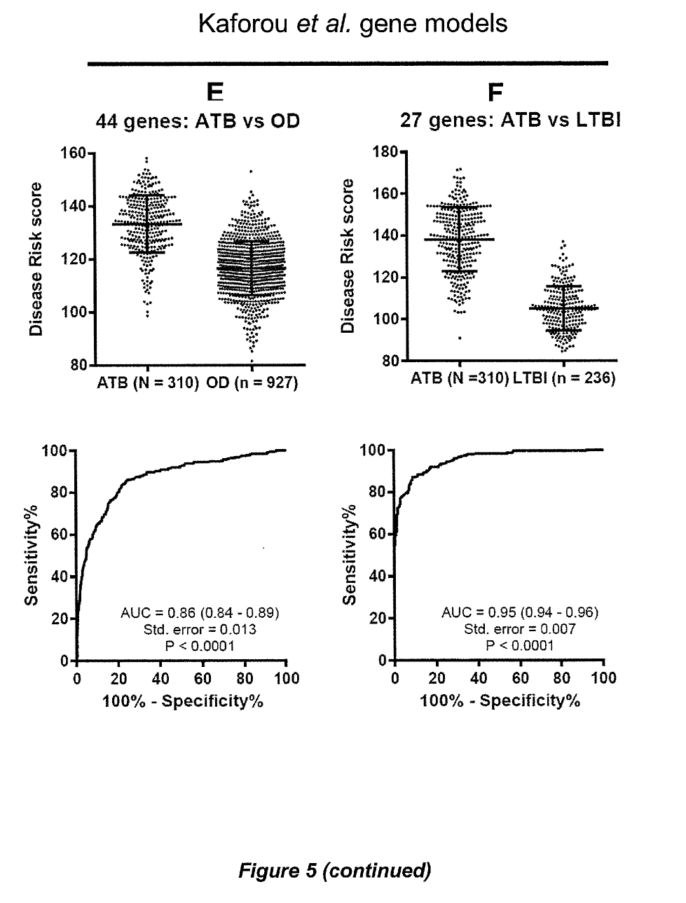

FIG. 5 shows that GBP6 and BATF2 could differentiate patients with ATB (N=310) from those with OD (N=861) or LTBI (236) with high accuracy even when HIV+ patients were included in the analysis

GBP6 was expressed at higher level in those with ATB relative to those with OD (A) or LTBI (B). An AUC value of 0.85 (0.83-0.88) was observed in ROC analysis for ATB vs. OD (A) and 0.90 (0.88-0.93) for ATB vs. LTBI (B). BATF2 was expressed at a higher level in those with ATB relative to those with OD (C) or with LTBI (D). An AUC value of 0.85 (0.83-0.88) was observed in ROC analysis for ATB vs. OD (C) and 0.90 (0.88-0.93) for ATB vs. LTBI (D). The diagnostic performance of gene modules described by Kaforou et al. [4] was tested on the combined dataset. The performance of 44 genes in distinguishing ATB from OD (E) and of 27 genes in distinguishing ATB from LTBI (F) were lower in this combined dataset in comparison with data reported.

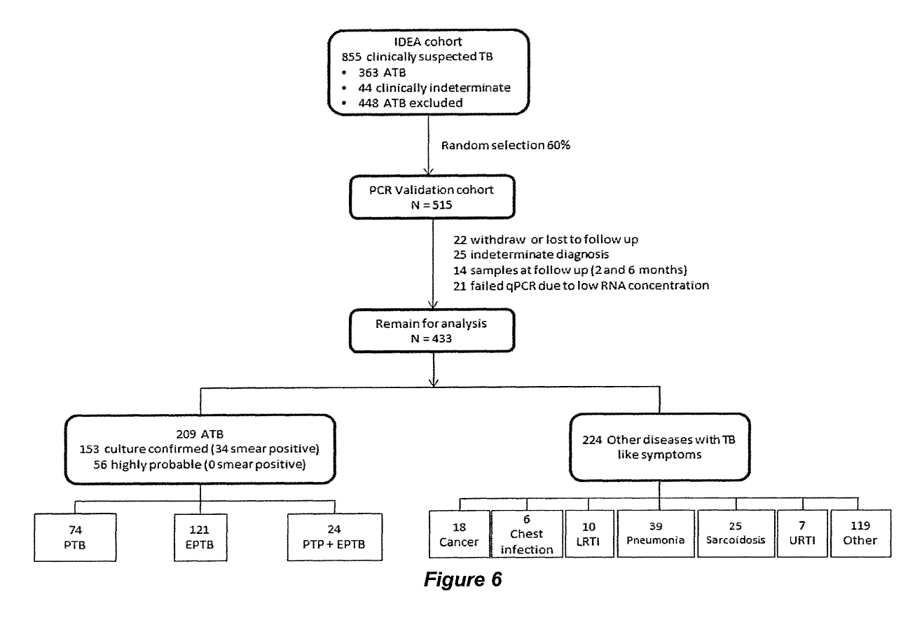

FIG. 6 shows a flow diagram of the samples selected from the IDEA cohort project for quantitative RT-PCR validation

From the total of 855 TB suspected cases, 515 samples with equal number of ATB and OD were randomly selected for validation. The operator who performed the quantitative PCR was blinded to the final diagnosis.

FIG. 7 shows the diagnostic performance of GBP6 and BATF2 in combination with TST

TST test result was available for 208 patients. 94/208 patients had ATB (Dasanjh category 1) and 114 had OD (Dosanjh Category 4). Patients who were diagnosed with probable TB were removed from this analysis (n=38). Patient was defined to be positive with TST test if the TST induration is ≥5 mm (for non BCG vaccinated patients) or ≥15 mm (for BCG vaccinated patients). (A) In patients who were positive with TST, GBP6 distinguished ATB from OD with 82.3% sensitivity (95CI: 72.5%-89.7%) and 85.1% specificity (95CI: 71.7%-93.8%). In patients who were negative with TST, the sensitivity and specificity were 100% (69.1%-100%) and 86.4% (75.7%-93.6%), respectively (Supp. FIG. 4A). B) BATF2 distinguished ATB from OD with 84.7% sensitivity (95CI: 75.3%-91.6%) and 82.2% specificity (95CI: 67.9%-92.0%). in patients who were positive with TST. In those who were negative with TST, BATF2 distinguished ATB from OD with 100% sensitivity (69.1%-100%) and 79.7% specificity (67.7%-88.7%).

### EXAMPLES

### Methods Used in the Examples

Published Data Normalisation and Combination

Raw microarray datasets of whole blood transcriptional profiles of adults with tuberculosis and other diseases were retrieved from the Gene Expression Omnibus database (GEO, http://www.ncbi.nlm.nih.gov/geo/) using the accession numbers GSE37250 [4], GSE42834 [12] and GSE19491 [3]. The transcriptional profiles of children with tuberculosis were retrieved using the accession number GSE39940 [6].

Both adult and paediatric datasets were normalised by the Lumi R package [13]. After normalisation, Principle Component Analysis (PCA) was performed to ensure the homogeneity of the data. To remove batch effects in the dataset, we employed ComBat R package [5]. To ensure the batch removal did not affect the integrity of the data, we randomly divided the healthy controls into two groups and performed t-tests to identify whether genes were differentially expressed between these groups. No differences were observed, indicating that the combination strategy used had not introduced artificial differences into the data.

Selection of Genes Combinations

To identify combinations of genes that could best discriminate ATB from OD, first, a T test was performed to identify genes differentially expressed between ATB and OD. By applying a False Discovery Rate cut-off at 0.001, a total 2,724 genes were identified. Next, 70% of the dataset was randomly selected as a training set (867 samples) and the remaining 30% as the test set (370 samples). The Nested (100 times) Random Forest [14] model was applied to the training data to calculate the mean importance score for each gene. Next, the top 30 genes with the highest mean importance scores were selected for a nested (1000 times) exhaustive subset selection [15] to identify optimal combinations of genes. Linear Discriminant Analysis (LDA) [16] with 10-fold cross validation was applied to check the performance of these combinations in the training data and validated in the test set. The R script of this analysis is as follows:

# Name: variable_selection_script.R
# Auth: Umar Niazi u.niazi@imperial.ac.uk
# Date: 05/01/2016
# Desc: script for variable selection
if(!require(downloader) ∥ !require(methods)) stop(‘Library downloader and methods
required’)

| | |
|---|---|
| url | = |

```
‘https://raw.githubusercontent.com/uhkniazi/CCrossValidation/master/CCrossValidati
on.R’
download(url, ‘CCrossValidation.R’)
# load the required packages
source(‘CCrossValidation.R’)
# delete the file after source
unlink(‘CCrossValidation.R’)
# utility function to load object
f_LoadObject = function(r.obj.file)
{
  # temp environment to load object
  env <− new.env( )
  # read object
  nm <− load(r.obj.file, env)[1]
  return(env[[nm]])
}
## load the test data
load(file=‘IData.publish.rds’)
# it is a list with various components
names(IData)
print(IData$desc)
## get the components from the list
# test set index
test = IData$test
# sample annotation
dfSamples = IData$sample
# expression matrix
mDat = IData$expression
# annotation data
dfAnnotation = IData$annotation
# set variables
dfSamples.train = dfSamples[−test,]
dfSamples.test = dfSamples[test,]
mDat.train = mDat[−test,]
mDat.test = mDat[test,]
## perform nested random forest on test set
## adjust boot.num as desired
dfData = as.data.frame(mDat.train)
oVar.r = CVariableSelection.RandomForest(dfData, dfSamples.train$fGroups.2,
boot.num = 100)
# plot the top 20 genes based on importance scort with 95% confidence interval for
standard error
plot.var.selection(oVar.r)
# get the variables
dfRF = CVariableSelection.RandomForest.getVariables(oVar.r)
# select the top 30 variables
cvTopGenes = rownames(dfRF)[1:30]
# use the top 30 genes to find top combinations of genes
dfData = as.data.frame(mDat.train)
dfData = dfData[,colnames(dfData) %in% cvTopGenes]
oVar.sub = CVariableSelection.ReduceModel(dfData, dfSamples.train$fGroups.2,
boot.num = 100)
# plot the number of variables vs average error rate
plot.var.selection(oVar.sub)
# print variable combinations
for (i in 2:6){
  cvTopGenes.sub = CVariableSelection.ReduceModel.getMinModel(oVar.sub, i)
  print(paste(‘Variable Count’, i))
  print(dfAnnotation[cvTopGenes.sub,])
}
## 10 fold nested cross validation with various variable combinations
par(mfrow=c(2,2))
# try models of various sizes with CV
for (i in 2:6){
  cvTopGenes.sub = CVariableSelection.ReduceModel.getMinModel(oVar.sub, i)
  dfData.train = as.data.frame(mDat.train)
  dfData.train = dfData.train[,colnames(dfData.train) %in% cvTopGenes.sub]
  dfData.test = as.data.frame(mDat.test)
  dfData.test = dfData.test[,colnames(dfData.test) %in% cvTopGenes.sub]
  oCV = CCrossValidation.LDA(test.dat = dfData.test, train.dat = dfData.train,
test.groups = dfSamples.test$fGroups.2,
```

train.groups = dfSamples.train$fGroups,2, level.predict = ‘ATB’,

```
boot.num = 500)
  plot.cv.performance(oCV)
  # print variable names and 95% confidence interval for AUC
  temp = oCV@oAuc.cv
  x = as.numeric(temp@y.values)
  print(paste(‘Variable Count’, i))
  print(dfAnnotation[cvTopGenes.sub,])
  print(signif(quantile(x, probs = c(0.025, 0.975)), 2))
}
```

Gene Selection Based on Detection P Value

For identification of biomarkers through gene expression data, the majority of the classification methods, such as support vector machine, random forest, neural network, shrunken centroids and LDA, are based on fluorescent signal values making. This makes combining datasets from different studies challenging due to technical and experimental variations. As with the Affymetrix platform, Illumina gene expression arrays provide both a quantitative fluorescent signal value and a qualitative value (detection P-value) for each gene. This value is complementary to the fluorescent signal and indicates if a gene has a certain probability of being detected in a sample. A detection P-value of ≤0.01 indicates the gene is consistently expressed and detected in each sample hybridized to the array. Importantly, this value is specific for each gene within each sample and is not affected when data from different studies are combined [17]. In the Affymetrix platform, a similar value was exploited and successfully used to develop a new class predictor method [18]. However, this important aspect of Illumina gene expression microarrays has not been investigated thoroughly. It was hypothesized that differences in gene detection rates between active TB (ATB), other diseases (OD) and latent TB infection (LTBI) could be exploited for biomarker identification. To discover genes that are over-expressed in ATB versus OD, we selected genes that were detected (P value≤0.01) in ≥90% of ATB patients and ≤50% of LTBI, OD and healthy controls and tested their ability to distinguish ATB from LTBI and OD in an independent cohort.

Validation Cohort

Whole blood samples were collected from patients with suspected ATB who were recruited as part of the “IGRA in Diagnostic Evaluation of Active TB (IDEA)”-study in 13 NHS Trust hospitals in London, Birmingham and Leicester (UK) from November 2011 to August 2013 (Ethics approval number 11/H0722/8/) (http://public.ukcrn.org.uk/search/StudyDetail.aspx?StudylD=10667). Informed consent was obtained from all participants. All the participants were recruited prospectively in secondary care before confirmatory diagnosis. A bio-bank of clinical samples, comprising whole blood samples, serum and PBMCs, was collected from these participants for the identification of novel biomarkers of active TB. Samples were collected at initial recruitment (baseline) and at follow up at two and six months to establish a final diagnosis. For the purpose of quantitative RT-PCR validation of our biomarkers, baseline samples from 161 active TB patients and 161 patients with OD were randomly selected.

Active TB was defined based on the Dosanjh categorisation [19] as: 1). Positive with microbiological culture of Mtb and suggestive clinical and radiological findings or 2) having clinical and radiological features highly suggestive of TB unlikely to be caused by other diseases, AND a decision to treat made by clinician, AND appropriate response to therapy, AND histological supportive evidence if available. LTBI was defined if a person was assigned to category 4 and was dual positive with either TST and QUANTIFERON Gold or TST and T-SPOT.TB. Other diseases cases were confirmed if they are classified as Dosanjh category 4 [19]. Finally, to avoid bias, the samples used for validation were randomly selected to include equal number of ATB and OD (including LTBI) and the operator who performed the qRT-PCR was completely blinded to the final diagnosis of the patients.

RNA Extraction Quantitative RT-PCR

RNA from whole blood samples was extracted from Paxgene RNA Blood tubes (Qiagen) with the Paxgene blood RNA extraction kit (Qiagen) according to the manufacturer's instructions. RNA QC was performed using both Nanodrop and Bioanalyser instruments. Only samples with an RNA integrity number (RIN) of >8 were used. A two-step qRT-PCR was performed to validate the microarray gene expression profiles of GBP6 and BATF2. Firstly, 250 ng of RNA was converted to cDNA using Maxima hexamer reverse transcriptase and random hexamer primers (Thermo). Quantitative PCR reactions, with 10 ng of cDNA, were performed using an ABI 7500 Fast machine using Taqman Gene Expression Assays (Life Technologies) for the target genes GBP6 (Hs01584201_m1), BATF2 (Hs00912737_m1). HPRT1 (Hs02800695_m1) was chosen as the house-keeping gene because its expression was consistent across all 1237 samples in the above-mentioned microarray dataset (data not shown). The relative abundances of each transcript was calculated using the delta-delta Ct methodology [20].

### Example 1—Identification of Biomarkers for ATB Diagnosis

Molecular biomarkers that contribute to TB diagnosis in the clinic, particular the challenge of differentiating ATB cases from LTBI with TB like symptoms are still lacking. It has been reported that the combined expression of IL-8, FOXP8, and IL-12b distinguishes ATB from TST+ healthcare workers [1] and expression of FCGR1B, LTF4, CD64 and GBP5 distinguishes ATB from healthy LTBI contacts [2]. Berry et al. [3] identified 383 genes, the majority of which were neutrophil-derived and type-I interferon regulated, distinguishing ATB from other diseases (OD) with 92% sensitivity and 83% specificity, and Kaforou et al. a set of 44 genes differentiating ATB from OD with 100% sensitivity and 96% specificity [4].

However, these case-control studies do not reflect the complexity of TB diagnosis in the clinic, particular the challenge of differentiating ATB cases from LTBI with TB like symptoms. Furthermore complex signatures based on combinations of many genes are difficult to incorporate into routine diagnosis [3, 4].

Nevertheless, these studies are a rich resource for data mining, consisting of transcriptomic data from hundreds of TB patient blood samples. It was hypothesised that analysis of a large combined dataset from published studies would facilitate identification of gene transcripts that might provide an improved diagnostic test for ATB.

Using a computational algorithm [5], the data from three independent adult transcriptomic studies was combined (see methods) into a dataset of transcripts from 1237 whole blood samples, comprising 310 ATB (176 HIV+), 66 sarcoidosis, 471 OD (176 HIV+), 236 LTBI and 154 healthy controls (HC) (FIG. 4). Principle Component Analysis (PCA) plots of the collated data indicate that study-related batch effects were effectively removed to generate a homogenous pattern (FIG. 1A-C).

To identify genes with high specificity for ATB compared to OD patients and LTBI, the gene detection rate P value measured in the Illumina platform, was applied as a cut-off (see methods). Although this value is generated as a standard quality control parameter, it has not been exploited for identification of disease biomarkers. In adults, GBP6 was detected in 90% of ATB patients but only 44%, 36% and 25% of LTBI, OD and HC, respectively (FIGS. 1D & E). Using GBP6 to distinguish ATB from OD generated a Receiver Operating Characteristic (ROC) curve with an Area Under the Curve (AUC) value of 0.85. A cut-off value of 7.06 distinguishes ATB from OD with 80% sensitivity and 79.5% specificity. For ATB vs LTBI the AUC value is 0.90 with 85.1% sensitivity (Table 2, FIGS. 5A and B).

TABLE 2
Diagnostic performance of GBP6 and BATF2 in adult gene expression microarray datasets

| | | GBP6 | | | | | BATF2 |
|---|---|---|---|---|---|---|---|
| | | AUC (95CI) | Sens (95CI) | Spec (95CI) | LR+ (95CI) | LR− (95CI) | AUC (95CI) |
| HIV+ and HIV− | ATB vs. | 0.85 | 80.0 | 79.5 | 3.90 | 0.25 | 0.85 |
| ATB = 310, | OD | (0.83-0.88) | (75.1-84.3) | (76.7-82.2) | (3.22-4.74) | (0.19-0.32) | (0.82-0.87) |
| LTBI = 236, | ATB vs. | 0.90 | 85.1 | 76.3 | 3.59 | 0.20 | 0.89 |
| OD = 861* | LTBI | (0.88-0.93) | (80.7-88.9) | (70.3-81.5) | (2.72-4.81) | (0.14-0.27) | (0.86-0.92) |
| HIV− | ATB vs. | 0.85 | 80.6 | 76.3 | 3.40 | 0.25 | 0.85 |
| ATB = 212, | OD | (0.82-84.3) | (74.7-85.7) | (73.0-79.5) | (2.77-4.18) | (0.18-0.35) | (0.82-0.86) |
| LTBI = 152, | ATB vs. | 0.93 | 90.4 | 76.3 | 3.81 | 0.13 | 0.93 |
| OD = 685* | LTBI | (0.90-0.95) | (86.4-94.5) | (68.7-82.8) | (2.76-5.49) | (0.07-0.20) | (0.91-0.96) |

| | | BATF2 | | | |
|---|---|---|---|---|---|
| | | Sens (95CI) | Spec (95CI) | LR+ (95CI) | LR− (95CI) |
| HIV+ and HIV− | ATB vs. | 80.0 | 76.3 | 3.56 | 0.26 |
| ATB = 310, | OD | (75.1-84.3) | (73.3-79.1) | (2.81-4.03) | (0.20-0.34) |
| LTBI = 236, | ATB vs. | 83.5 | 80.1 | 4.30 | 0.21 |
| OD = 861* | LTBI | (78.94-85.5) | (74.4-84.9) | (3.08-5.66) | (0.17-0.28) |
| HIV− | ATB vs. | 80.6 | 79.3 | 4.14 | 0.24 |
| ATB = 212, | OD | (74.7-85.7) | (76.0-82.3) | (3.11-4.84) | (0.17-0.33) |
| LTBI =152, | ATB vs. | 92.4 | 76.3 | 4.03 | 0.10 |
| OD = 685* | LTBI | (88-95.6) | (68.7-82.8) | (2.81-5.56) | (0.05-0.17) |

TABLE 3
Diagnostic performance of GBP6 and BATF2 in children gene expression microarray datasets

| | | GBP6 | | | | | BATF2 |
|---|---|---|---|---|---|---|---|
| | | AUC (95CI) | Sens (95CI) | Spec (95CI) | LR+ (95CI) | LR− (95CI) | AUC (95CI) |
| HIV+ and HIV− | ATB vs. | 0.85 | 80.2 | 78.9 | 3.80 | 0.25 | 0.82 |
| ATB = 111, | OD | (0.80-0.89) | (71.5-87.0) | (73.0-84.0) | (2.65-5.44) | (0.15-0.39) | (0.77-0.88) |
| LTBI = 54, | ATB vs. | 0.86 | 82.0 | 81.5 | 4.40 | 0.22 | 0.85 |
| OD = 223* | LTB | (0.81-0.92) | (73.5-88.6) | (68.6-90.7) | (2.34-9.53) | (0.13-0.39) | (0.80-0.91) |
| HIV− | ATB vs. | 0.88 | 84.3 | 80.2 | 3.90 | 0.20 | 0.83 |
| ATB = 70. | OD | (0.83-0.93) | (73.6-91.9) | (73.6-86.1) | (2.79-6.61) | (0.09-0.36) | (0.77-0.89) |
| LTBI = 54, | ATB vs. | 0.88 | 84.3 | 84.4 | 3.80 | 0.19 | 0.84 |
| OD = 157 | LTB | (0.82-0.94) | (73.6-91.9) | (68.5-90.7) | (2.34-9.88) | (0.09-0.39) | (0.77-0.90) |

| | | BATF2 | | | |
|---|---|---|---|---|---|
| | | Sens (95CI) | Spec (95CI) | LR+ (95CI) | LR− (95CI) |
| HIV+ and HIV− | ATB vs. | 77.5 | 76.2 | 3.56 | 0.30 |
| ATB = 111, | OD | (68.6-84.8) | (70.0-81.6) | (2.29-4.61) | (0.19-0.45) |
| LTBI = 54, | ATB vs. | 80.2 | 77.8 | 3.92 | 0.25 |
| OD = 223* | LTB | (71.5-87.1) | (64.4-87.9) | (2.01-7.20) | (0.15-0.44) |
| HIV− | ATB vs. | 77.1 | 75.1 | 3.47 | 0.30 |
| ATB = 70. | OD | (65.5-86.3) | (67.6-81.7) | (2.02-4.72) | (0.17-0.51) |
| LTBI = 54, | ATB vs. | 78.5 | 77.8 | 3.94 | 0.28 |
| OD = 157 | LTB | (67.1-87.4) | (64.4-87.9) | (1.88-7.22) | (0.14-0.51) |

*other diseases group including healthy controls and LTBI subjects;
AUC: Area Under the Curve;
Sens: Sensitivity;
Spec: Specificity;
95CI: 95% confidence interval

The ROC was largely unaffected by HIV status and HIV− samples gave an AUC of 0.85 with 80.6% sensitivity and 76.3% specificity for ATB vs OD and an AUC of 0.93 with 90.4% sensitivity and 76.3% specificity for ATB vs LTBI (Table 2, FIGS. 2A and B).

We also identified a second gene (BATF2) detected in 90% of ATB but <50% of LTBI, HC and OD patients (FIG. 1F). BATF2 could distinguish ATB from OD and LTBI with similar performance metrics to GBP6 (Table 2, FIGS. 2C and D). Interestingly, a single gene (GBP6 or BATF2) applied to the combined dataset, gave a diagnostic performance similar to that of the 44 genes for ATB vs OD or 27 genes for ATB vs LTBI described in Kaforou et al., in which a Disease risk score was used (FIGS. 5E and F).

### Example 2—Diagnostic Performance of GBP6 and BATF2

The diagnostic performance of GBP6 and BATF2 for paediatric patients was investigated in a published dataset comprising 334 whole blood samples (111 ATB, 54 LTBI and 169 OD) [6]. The results are summarised in Table 3. When HIV+ patients were included, GBP6 gave 80.2% sensitivity and 78.9% specificity for ATB vs OD and 82.0% sensitivity and 81.5% specificity for ATB vs LTBI. When HIV+ subjects were excluded, GBP6 distinguished ATB from OD with 84.3% sensitivity and 80.2% specificity and 84.3% sensitivity and 84.4% specificity for ATB vs LTBI. BATF2 distinguishes children with ATB from those with OD or LTBI with sensitivity and specificity less than 80%. Notably, GBP6 or BATF2 performed as well as the disease risk score derived from 51 transcripts applied to the same patient cohort [6].

Random Forest and Linear Discriminant Analysis (LDA) (see methods) identified the best combinations of genes distinguishing ATB from OD (Table 4). However, the diagnostic performance of these combinations was no better than GBP6 or BATF2 alone. As a result, GBP6 and BATF2 were selected for validation by qRT-PCR.

TABLE 4
AUC values observed for GBP6 or BATF2 in comparison with different
gene combinations determined by Random Forest and LDA.

| Method | Models | AUC (95CI) |
|---|---|---|
| Detection P value | GBP6 | 0.86 (0.83-0.90) |
| Detection P value | BATF2 | 0.84 (0.80-0.88) |
| Model 1 | GBP5, DUSP3 | 0.85 (0.83-0.87) |
| Model 2 | DUSP3, WDFY1, GBP6 | 0.85 (0.83-0.88) |
| Model 3 | GBP5, DUSP3, ASGR2, GBP6 | 0.86 (0.84-0.88) |
| Model 4 | GBP5, DUSP3, JAK1, ASGR2, | 0.86 (0.84-0.88) |
| | AFF1 | |
| Model 5 | GBP5, DUSP3, JAK1, ASGR2, | 0.86 (0.84-0.88) |
| | AFF1, GBP6 | |

To investigate the performance of these genes in clinical TB diagnosis the mRNA abundance of GBP6 and BATF2 was measured using quantitative RT-PCR in an independent cohort of UK patients with suspected TB at the time of recruitment. This cohort included 209 ATB and 224 OD patients (43 with LTBI) (Table 5).

TABLE 5
Baseline demographic and clinical characteristics
of patients in the validation cohort.

| Characteristics | ATB (N = 212) | | OD (N = 222) | | P value* |
|---|---|---|---|---|---|
| Age, median (IQR), years | 32 | (16) | 42 | (23.3) | <0.0001 |
| Sex, male (%) | 134 | (63.2) | 133 | (59.9) | NS |
| Ethnicity, n (%) | | | | | |
| Indian subcontinent | 157 | (74.1) | 120 | (54.1) | |
| Black | 28 | (13.2) | 25 | (15.8) | |
| White | 15 | (7.1) | 42 | (18.9) | <0.0001 |
| Asian | 10 | (4.7) | 10 | (4.5) | |
| Middle eastern | 1 | (0.5) | 8 | (3.6) | |
| Other | 1 | (0.5) | 7 | (3.2) | |
| BMI (kg/m²), | 22.1 | (4.9) | 24.7 | (7.5) | <0.0001 |
| median (IQR) | | | | | |
| BCG vaccination, n (%) | 153 | (72.2) | 171 | (77) | NS |
| BCG scar, n (%) | 136 | (64.2) | 143 | (64.4) | NS |
| TB contact, n (%) | 55 | (25.9) | 50 | (22.5) | NS |
| Symptoms | | | | | |
| Fever, n (%) | 106 | (52.5) | 92 | (41.4) | <0.05 |
| Cough, n (%) | 143 | (70.8) | 154 | (69.4) | NS |
| Night sweat, n (%) | 104 | (51.5) | 113 | (50.9) | NS |
| Weight loss, n (%) | 113 | (55.9) | 107 | (48.2) | NS |
| Haemoptysis, n (%) | 28 | (13.9) | 34 | (15.3) | NS |
| Lethargy, n (%) | 107 | (53) | 117 | (52.7) | NS |
| Other, n (%) | 100 | (47.1) | 109 | (49) | NS |

Footnote:
NS: not significant

TABLE 6
Final diagnosis of LTBI patients

| | Age | | | TST | BCG | | | |
|---|---|---|---|---|---|---|---|---|
| SampleID | (years) | Sex | Diag-sis | mm | scar | TST +/− | T-SPOT +/− | QFN +/− |
| A131 | 46 | Female | Previous treated TB, post TB bronchiectisis | ND | + | NA | + | + |
| H001 | 20 | Male | Cancer | ND | + | NA | + | + |
| N030 | 36 | Female | BILATERAL PLEURAL EFFUSION | 20 | + | + | + | − |
| G004 | 73 | Male | ATYPICAL MYCOBACTERIUM INFECTION | 15 | + | + | + | − |
| A069 | 74 | Male | Chest Infection, Other | ND | + | NA | + | + |
| N115 | 34 | Male | Previous treated TB, Old TB scarring | ND | + | NA | + | + |
| A070 | 62 | Male | LTBI - treatment indicated | 15 | − | + | + | − |
| A078 | 52 | Male | Post thoracotomy pleural effusion | ND | + | NA | + | + |
| N132 | 65 | Male | Cancer | 35 | + | + | + | + |
| N126 | 36 | Female | URTI | 36 | + | + | − | + |
| A052 | 51 | Male | Cancer | ND | + | NA | + | + |
| N145 | 73 | Male | Testicular nodularity, epidimitis, | ND | − | NA | + | + |
| | | | old self heale TB | | | | | |
| A075 | 67 | Female | Chronic lung scarring | 46 | + | + | + | + |
| A270 | 71 | Male | Other, Sarcoidosis | 18 | + | + | − | + |
| G017 | 73 | Female | Other | 15 | + | + | − | + |
| G013 | 27 | Female | LTBI - treatment indicated, Other | 15 | + | + | + | − |
| N153 | 33 | Male | LTBI - treatment indicated | 18 | − | + | − | + |
| A208 | 30 | Female | Other | 17 | + | + | + | + |
| N210 | 27 | Male | LTBI - treatment indicated, Other, URTI | 39 | + | + | + | + |
| N157 | 52 | Female | Other | 30 | + | + | + | + |
| S001 | 30 | Female | ASTHMA | ND | − | NA | + | + |
| N270 | 52 | Male | Upper Respiratory Tract Infection | 25 | − | + | + | + |
| B032 | 29 | Male | Normal | ND | + | NA | + | + |
| A124 | 38 | Male | SELF-RESOLVING upperlobe lesion | ND | + | NA | + | + |
| A048 | 23 | Female | Self resolving haemoptysis | 21 | + | + | + | + |
| L102 | 60 | Male | Atypical mycobacterium infection of | ND | + | NA | + | + |
| | | | corneal graft | | | | | |
| A155 | 60 | Male | anthracosis | ND | + | NA | + | + |
| L036 | 41 | Female | rheumatoid arthritis | ND | + | NA | + | + |
| L054 | 19 | Male | Vascular eye disease | ND | − | NA | + | + |
| B089 | 54 | Male | self-resolving cough | ND | − | NA | + | + |
| N266 | 28 | Male | Upper Respiratory Tract Infection | 28 | − | + | + | + |
| N022 | 33 | Male | LTBI - treatment indicated | 22 | + | + | + | + |
| L017 | 61 | Male | Renal cell carci-ma | ND | + | NA | + | + |
| | | | LTBI - - treatment indicated | | | | | |
| A117 | 39 | Female | Lower Respiratory Tract Infection | 20 | − | + | + | + |
| A264 | 33 | Female | non specific panuveitis | 17 | − | + | + | + |
| A157 | 51 | Female | Breast Cancer LTBI (treatment indicated) | 18 | − | + | + | + |
| B046 | 42 | Male | Benign skin lesion | ND | + | NA | + | + |
| A120 | 43 | Male | Pneumonia | ND | + | NA | + | + |
| N291 | 30 | Female | Cervical lymphade-pathy | ND | − | NA | + | + |
| B059 | 33 | Male | LTBI - treatment indicated | 38 | + | + | + | + |
| A244 | 72 | Male | community acquired pneumonia | ND | − | NA | + | + |
| N021 | 33 | Male | LTBI - treatment indicated | 21 | + | + | + | + |
| N134 | 80 | Male | RENAL PROBLEMS with kiebsiella bacteraemia | ND | − | NA | + | + |

ND: not done;
URTI Upper respiratory tract infection;
QTF quantiferon gold in tube;
TSPOT: Elispot-based IGRA test

The final diagnoses of ATB and OD patients are summarised in FIG. 6 and of LTBI patients in Table 6.

We analysed the data with and without 25 patients with sarcoidosis. mRNA levels of GBP6 and BATF2 identified by qPCR were significantly higher in patients with ATB vs OD and LTBI (FIG. 3). This difference was not confounded by age, BMI and ethnicity (Tables 7 & 8).

TABLE 7
Univariant logistic regression: GBP6
is an independent predictor for ATB.

| | | | | Odds | |
|---|---|---|---|---|---|
| | | | P | Ratio | 95% CI |
| Predictor* | β | S.E | value | (OR) | for OR |
| Age | −0.014 | 0.011 | 0.185 | 0.986 | 0.966-1.007 |
| Indian subcontinent | Ref. | | | | |
| Black | −0.275 | 0.444 | 0.536 | 0.760 | 0.318-1.813 |
| White | −1.079 | 0.499 | 0.031 | 0.340 | 0.128-0.904 |
| Asian | 0.449 | 0.700 | 0.521 | 1.567 | 0.397-6.185 |
| Middle eastern | −2.080 | 1.090 | 0.080 | 0.113 | 0.013-0.957 |
| BMI | −0.062 | 0.029 | 0.032 | 0.940 | 0.889-0.995 |
| GBP6 | 0.130 | 0.034 | <0.0001 | 1.143 | 1.069-1.221 |

*Model parameters: constant: 1.472, −2Log likelihood 230 −Chi2 <0.0001, Hosmer and lemeshow test 0.27, R2 (Cox 0.275 and Nagelkerke 0.344).

TABLE 8
Univariant logistic regression: BATF2
is an independent predictor for ATB.

| | | | | Odds | |
|---|---|---|---|---|---|
| | | | P | Ratio | 95% CI |
| Predictor* | β | S.E | value | (OR) | for OR |
| Age | −0.013 | 0.011 | 0.245 | 0.987 | 0.966-1.009 |
| Indian subcontinent | Ref. | | | | |
| Black | −0.268 | 0.463 | 0.563 | 0.765 | 0.309-1.896 |
| White | −0.966 | 0.510 | 0.058 | 0.381 | 0.140-1.034 |
| Asian | 0.566 | 0.716 | 0.429 | 1.762 | 0.433-7.168 |
| Middle eastern | −2.613 | 1.226 | 0.033 | 0.073 | 0.007-0.811 |
| BMI | −0.047 | 0.029 | 0.110 | 0.954 | 0.900-1.011 |
| BATF2 | 0.164 | 0.034 | <0.0001 | 1.178 | 1.103-1.259 |

*Model parameters: constant: 0.765, −2Log likelihood 217 −Chi2 <0.0001, Hosmer and lemeshow test 0.32, R2 (Cox 0.300 and Nagelkerke 0.401).

GBP6 distinguished ATB from OD with 81.3% sensitivity and 83.4% specificity and positive and negative likelihood ratios (LRs) of 4.9 and 0.22, respectively while for ATB vs LTBI the sensitivity, specificity, positive and negative LRs were 83.7%, 84.1%, 5.3 and 0.19, respectively (Table 1 and FIGS. 3A and B). BATF2 gave slightly lower sensitivity, specificity and likelihood ratios (Table 1 and FIGS. 3C and D). Multivariate analysis revealed that GBP6 and BATF2 are independent predictors for ATB; Odds Ratio (OR) 1.143 (95% CI 1.069-1.221) and 1.178 (95% CI 1.103-1.259) respectively, but as the expression level of GBP6 and BATF2 are highly correlated, a predictive model combining both genes did not improve diagnostic sensitivity (data not shown).

It was tested whether combining GBP6 and BATF2 with existing diagnostic tests might improve diagnostic performance. Combining the new markers with T-SPOT did not improve sensitivity or specificity in distinguishing TB from OD (data not shown). In contrast, when combined with TST, our markers improved the sensitivity and specificity significantly (FIG. 7). In TST+, patients GBP6 distinguished ATB from OD with 82.3% sensitivity (95CI: 72.5%-89.7%) and 85.1% specificity (95CI: 71.7%-93.8%). In patients who were TST−, the sensitivity and specificity were 100% (69.1%-100%) and 86.4% (95CI: 75.7%-93.6%), respectively (FIG. 7A). Similarly, BATF2 improved the sensitivity and specificity of diagnosis in patients who were TST+ or TST− (FIG. 7B).

A minor limitation in the use of GBP6 and BATF2 as biomarkers for ATB is in sarcoidosis where the expression levels are similar to ATB, however other blood transcripts may distinguish ATB from this disease.

Diagnosis of sarcoidosis can be based on exclusion of ATB such as the negative test results for ATB (smear, culture and PCR). Individuals with sarcoidosis often do not respond to TB treatments. Histopathology can also be used to investigate the granulomas in sarcoidosis.

GBP6 is an IFNγ-stimulated gene that plays a role in host defence against mycobacteria in mice [7] and in macrophages in vitro, together with GBP1 and GBP10 [7, 8]. BATF2 is strongly induced in IFNγ-activated and Mtb infected macrophages with increased expression of BATF2-dependent genes such as TNF, CCL5, IL12B, and NOS2 [9]. BATF family genes interact with interferon-regulatory factor (IRF) genes [10]. Higher expression of GBP6 and BATF2 in ATB patients may be due to the higher mycobacterial burden and interferon response in ATB compared to LTBI patients. High expression of GBP6 and BATF2 in some LTBI patients may indicate an increased risk of developing ATB. To confirm this, the expression level of GBP6 and BATF2 will be investigated in patients who were the close contacts of ATB cases and were negative with IGRAs test but subsequently progressed to ATB.

### Discussion of Results of Examples 1-2

Distinguishing ATB from other diseases with TB-like symptoms and LTBI is challenging. Here, we identified two biomarkers using an unprecedented detection P value approach in the largest genome-wide transcriptomic dataset available to date.

We demonstrate first, that the peripheral blood mRNA levels of GBP6 and BATF2 differentiate ATB from LTBI patients with TB-like symptoms with ˜80% sensitivity and high specificity and are a significant improvement over culture and sputum smear microscopy, which have a sensitivity of ˜65% [11]. Second, expression of GBP6 or BATF2 qPCR improves sensitivity in detecting ATB in those who are TST+ and, in TST− patients, low expression might be used as a rule-out test. Third, these markers were validated in a cohort recruited in routine clinical practice reflecting the real-life spectrum of patients presenting with suspected active TB, whereas previous case-control studies included only culture-confirmed TB and LTBI subjects who were otherwise healthy [3, 4]. Fourth, the sensitivity of GBP6 or BATF2 in distinguishing ATB from OD is comparable to that of IGRA but unlike IGRA, GPB6 and BATF2 can also distinguish ATB from LTBI. Lastly, the expression level of a single gene can distinguish ATB from OD, increasing the likelihood of developing a test that could be used in low-income settings. GBP6 and BATF2 are excellent biomarkers of ATB and expected to be clinically valuable.

The listing or discussion of an apparently prior-published document in this specification should not necessarily be taken as an acknowledgement that the document is part of the state of the art or is common general knowledge.

### REFERENCES

1. Wu, B., et al., Messenger RNA Expression of IL-8, FOXP3, and IL-12R Differentiates Latent Tuberculosis Infection from Disease. Journal of Immunology, 2009. 178(6): p. 3688-3694.
2. Maertzdorf, J., et al., Human gene expression profiles of susceptibility and resistance in tuberculosis. Genes and immunity, 2011. 12(1): p. 15-22.
3. Berry, M. P. R., et al., An interferon-inducible neutrophil-driven blood transcriptional signature in human tuberculosis. Nature, 2010. 466(7309): p. 973-977.
4. Kaforou, M., et al., Detection of Tuberculosis in HIV-Infected and -Uninfected African Adults Using Whole Blood RNA Expression Signatures: A Case-Control Study. PLoS Medicine, 2013. 10.
5. Johnson, W. E., C. Li, and A. Rabinovic, Adjusting batch effects in microarray expression data using empirical Bayes methods. Biostatistics, 2007. 8(1): p. 118-27.
6. Anderson, S. T., et al., Diagnosis of childhood tuberculosis and host RNA expression in Africa. N Engl J Med, 2014. 370(18): p. 1712-23.
7. Degrandi, D., et al., Extensive characterization of IFN-induced GTPases mGBP1 to mGBP10 involved in host defense. J Immunol, 2007. 179(11): p. 7729-40.
8. Kim, B. H., et al., A family of IFN-gamma-inducible 65-kD GTPases protects against bacterial infection. Science, 2011. 332(6030): p. 717-21.
9. Roy, S., et al., Batf2/Irf1 Induces Inflammatory Responses in Classically Activated Macrophages, Lipopolysaccharides, and Mycobacterial Infection. J Immunol, 2015. 194(12): p. 6035-44.
10. Murphy, T. L., R. Tussiwand, and K. M. Murphy, Specificity through cooperation: BATF-IRF interactions control immune-regulatory networks. Nat Rev Immunol, 2013. 13(7): p. 499-509.
11. Agency, H. P., Tuberculosis in the UK: Annual report on tuberculosis surveillance in the UK, 2012.
2012.
12. Bloom, C. I., et al., Transcriptional Blood Signatures Distinguish Pulmonary Tuberculosis, Pulmonary Sarcoidosis, Pneumonias and Lung Cancers. PLoS ONE, 2013. 8(8).
13. Du, P., W. A. Kibbe, and S. M. Lin, lumi: a pipeline for processing Illumina microarray. Bioinformatics, 2008. 24(13): p. 1547-8.
14. Liaw, A. and M. Wiener, Classification and Regression by randomForest. R News, 2002. 2(3): p. 5.
15. Miller, T. L.u.F.c.b.A., leaps: regression subset selection. 2009.
16. Venables, W. N. and B. D. Ripley, Modern Applied Statistics with S. Springer, 2002. ISBN 0-387-95457-0.
17. Archer, K. J. and S. E. Reese, Detection call algorithms for high-throughput gene expression microarray data. Brief Bioinform, 2010. 11(2): p. 244-52.
18. Reme, T., et al., A new method for class prediction based on signed-rank algorithms applied to Affymetrix microarray experiments. BMC Bioinformatics, 2008. 9: p. 16.
19. Dosanjh, D. P., et al., Improved diagnostic evaluation of suspected tuberculosis. Ann Intern Med, 2008. 148(5): p. 325-36.
20. Livak, K. J. and T. D. Schmittgen, Analysis of relative gene expression data using real-time quantitative PCR and the 2(-Delta Delta C(T)) Method. Methods, 2001. 25(4): p. 402-8.

1: A method for diagnosing active tuberculosis (ATB) in an individual comprising the steps of:
a) providing a sample to be tested from the individual; and
b) measuring the expression in the test sample of GBP6 and/or BATF2;
wherein the expression in the test sample of GBP6 and/or BATF2 is indicative of the presence of active tuberculosis (ATB) in the individual.

2: The method according to claim 1 further comprising the steps of:
c) providing a control sample from an individual not afflicted with active tuberculosis (ATB); and
d) measuring the expression in the control sample of GBP6 and/or BATF2;
wherein the presence of ATB is identified in the event that the expression in the test sample of GBP6 and/or BATF2 measured in step (b) is higher than the expression in the control sample of GBP6 and/or BATF2 measured in step (d).

3: The method according to claim 1 further comprising the steps of:
c) providing a control sample from an individual afflicted with active tuberculosis (ATB); and
d) measuring the expression in the control sample of GBP6 and/or BATF2;
wherein the presence of ATB is identified in the event that the expression in the test sample of GBP6 and/or BATF2 measured in step (b) corresponds to the expression in the control sample of GBP6 and/or BATF2 measured in step (d).

4. (canceled)

5: A method for determining the risk of a latent tuberculosis infection (LTBI) progressing to active tuberculosis (ATB) in an individual comprising the steps of:
(a) providing a sample to be tested from the individual; and
(b) measuring the expression in the test sample of GBP6 and/or BATF2;
wherein the expression in the test sample of GBP6 and/or BATF2 is proportional to the risk of a latent TB infection progressing to an active TB infection in the individual.

6: The method according to claim 5, wherein a higher expression level of GBP6 and/or BATF2 measured in step (b) correlates with an increased risk of a latent TB infection progressing to an active TB infection.

7: The method according to claim 1, wherein the expression of an additional one or more biomarkers is measured alongside the expression of GBP6 and/or BATF2.

8: The method according to claim 7, wherein the additional one or more biomarkers does not include ANKRD22; FCGR1A; SERPTNG1; FCGR1C; FCGR1B; LOC728744; IFITM3; EPSTI1; GBP5; IFI44L; GBP1; LOC400759; IFIT3; AIM2; SEPT4; C1QB; GBP1; RSAD2; RTP4; CARD 17; IFIT3; CASP5; CEACAM1; CARD 17; ISG15; IFI27; TIMM10; WARS; IFI6; TNFAIP6; PSTPIP2; IFI44; SC02; FBX06; FER1L3; CXCL10; DHRS9; OAS1; STAT1; HP; DHRS9; CEACAM1; SLC26A8; CACNA1E; OLFM4; or APOL6.

9: The method according to claim 7, wherein the expression of no more than an additional 4 biomarkers is measured alongside the expression of GBP6 and/or BATF2.

10: The method according to claim 1, wherein the expression of only GBP6 and/or BATF2 is measured in any step.

11-13. (canceled)

14: The method according to claim 1 further comprising the additional step of performing the Tuberculosis Skin Test (TST) or providing results from a Tuberculosis Skin Test (TST) which has been performed on the individual; optionally wherein the presence of ATB is confirmed in the event that the Tuberculosis Skin Test (TST) or result thereof is positive.

15: The method according to claim 1, wherein the expression in the test sample of GBP6 and/or BATF2 is indicative of the presence of active tuberculosis (ATB) in the individual if the relative abundance expression is higher than 1.59 for GBP6 and/or 3.37 for BATF2

16: The method according to claim 1, wherein the individual does not have sarcoidosis.

17: The method according to claim 1 wherein the sample is a blood sample or PBMC sample.

18: The method according to claim 1, wherein step (b) comprises measuring the expression of a nucleic acid molecule encoding GBP6 and/or BATF2.

19-21. (canceled)

22: The method according to claim 1, wherein measuring the expression of GBP6 and/or BATF2 in step (b) is performed using one or more binding moieties, each individually capable of binding selectively to a nucleic acid molecule encoding GBP6 and/or BATF2.

23. (canceled)

24: The method according to claim 1, wherein step (b) is performed using a first binding agent capable of binding to GBP6 and/or BATF2.

25: The method according to claim 24 wherein the first binding agent comprises an antibody or an antigen-binding fragment thereof.

26-30. (canceled)

31: The method according to claim 1, wherein the method is at least 80% sensitive;
wherein the method is at least 80% specific; and/or
wherein the predicative accuracy of the method, as determined by an ROC AUC value, is at least 0.80.

32-34. (canceled)

35: The method according to claim 1, wherein the method comprises the additional step of identifying the most appropriate treatment for the individual and/or providing the individual with ATB treatment.

36-40. (canceled)

41: A kit for determining either the presence of an ATB infection in an individual, or for determining the risk of a latent tuberculosis infection (LTBI) progressing to ATB in an individual, comprising:
A) one or more binding agents each individually capable of binding selectively to a nucleic acid molecule encoding GBP6 and/or BATF2 or each individually capable of binding to GBP6 and/or BATF2; and
B) instructions for performing the method.

42. (canceled)

43. (canceled)
